(19) United States
(12) Patent Application Publication
(10) Pub. No.: US 2019/0304580 A1
(43) Pub. Date: Oct. 3, 2019

(54) TECHNOLOGIES FOR FACILITATING THE GENERATION AND DISTRIBUTION OF A DRUG FORMULARY

(71) Applicant: Formulary Academy, LLC, Carmel, IN (US)

(72) Inventors: Amy Renae Heath, Carmel, IN (US); Peter Anthony Vaccaro, Andover, MA (US)

(21) Appl. No.: 16/365,051

(22) Filed: Mar. 26, 2019

Related U.S. Application Data
(60) Provisional application No. 62/648,635, filed on Mar. 27, 2018.

(51) Int. Cl. G16H 15/00; G16H 70/40; G16H 20/10; G06F 17/24; G06F 16/93

(52) U.S. Cl. CPC G16H 15/00; G16H 70/40; G06F 16/93; G06F 17/24; G16H 20/10

(57) ABSTRACT

A method for generating a drug formulary or monograph including publicly available information and privately available information. A plurality of the generated monographs are stored and useful information is extracted from each of the monographs to improve patient healthcare outcomes. The extracted information provides an insight into the requested use or actual use by a patient or a medical facility of a prescription or non-prescription drug. For instance, the frequency of certain types of drug monographs could indicate a trend in the treatment of certain ailments and diseases. The drug formularies could indicate regions of the country where certain drugs are more often prescribed or requested. This indicator could be used by a drug manufacturer to increase production of a certain drug or increase the delivery to a certain region, or by a healthcare practitioner to determine which disease conditions or ailments occur more often and which require increased monitoring.

### CROSS-REFERENCE TO RELATED APPLICATIONS

This application claims the benefit of U.S. Provisional Application No. 62/648,635 filed on Mar. 27, 2018, the contents of which are incorporated herein by reference in their entirety.

### BACKGROUND

A drug formulary is a collection of documents identifying and detailing the characteristics of prescription drugs, including generic and brand name drugs, used by practitioners, such as doctors, nurse practitioners, and pharmacists, and other stakeholders. The drug formulary includes information describing each drug in sufficient detail to assist the practitioner in identifying a specific drug that offers the greatest overall value in effectively treating a particular condition. A committee including physicians, nurse practitioners, and pharmacists maintain the formulary. Non-prescription drugs can also be the subject of a drug formulary.

The rising costs of drugs has spawned an equally aggressive trend by managed pharmacy insurance plans to contain these costs via formulary benefit designs and restrictions. For example, closely managed pharmacy plans currently spend nearly thirty percent (30%) less per member on traditional medications, when compared to unmanaged plans. As a result, many or most existing pharmacy benefit plans are becoming more restrictive.

In order to more closely manage pharmacy plans, insurers are required to complete drug class reviews to provide clinical support to such decisions. This is a tedious process requiring the collection and assembly of disparate information from numerous different online sources. Accordingly, a technology is need to, among other things, assemble and provide a drug formulary product taking into account disparate information from several sources, so formulary reviews can be performed on the same information, but without the manual assembly of that information.

### SUMMARY

According to an embodiment, a method for generating healthcare information based on a plurality of drug monographs includes retrieving healthcare information from one or more databases, generating a plurality of drug monographs using the healthcare information retrieved from the one or more databases and based on user input from a plurality of users, monitoring activity of the plurality of users during the generation of the plurality of drug monographs, and generating a report indicating a pattern of usage associated with one or more of the plurality of drug monographs based on the monitored activity of the plurality of users. In some embodiments, the pattern of usage may include trend and occurrence information associated with one or more of the plurality of drug monographs. In some embodiments, monitoring the activity of the plurality of users during the generation of the plurality of drug monographs may include identifying a frequency of one or more studies being included, excluded, viewed, or not viewed in preparation of the plurality of drug monographs. In some embodiments, the one or more studies may include one or more client or competitor studies. In some embodiments, monitoring the activity of the plurality of users during the generation of the plurality of drug monographs may include identifying a geographic region associated with each user generating a drug monograph. In some embodiments, generating the report indicating the pattern of usage associated with one or more of the plurality of drug monographs may include indicating a pattern of usage associated with the healthcare information. In some embodiments, the pattern of usage may include identifying a frequency of generation of drug monographs for a particular drug.

According to another embodiment, a server for generating healthcare information based on a plurality of drug monographs includes a processor and a memory comprising a plurality of instructions stored thereon that, in response to execution by the processor, causes the server to retrieve healthcare information from one or more databases, generate a plurality of drug monographs using the healthcare information retrieved from the one or more databases and based on user input from a plurality of users, monitor activity of the plurality of users during the generation of the plurality of drug monographs, and generate a report indicating a pattern of usage associated with one or more of the plurality of drug monographs based on the monitored activity of the plurality of users. In some embodiments, the pattern of usage may include trend and occurrence information associated with one or more of the plurality of drug monographs. In some embodiments, to monitor the activity of the plurality of users during the generation of the plurality of drug monographs may include to identify a frequency of one or more studies being included, excluded, viewed, or not viewed in preparation of the plurality of drug monographs. In some embodiments, the one or more studies may include one or more client or competitor studies. In some embodiments, to monitor the activity of the plurality of users during the generation of the plurality of drug monographs may include to identify a geographic region associated with each user generating a drug monograph. In some embodiments, to generate the report indicating the pattern of usage associated with one or more of the plurality of drug monographs may include to indicate a pattern of usage associated with the healthcare information. In some embodiments, the pattern of usage may include identification of a frequency of generation of drug monographs for a particular drug.

According to yet another embodiment, one or more non-transitory machine-readable media comprising a plurality of instructions stored thereon that, in response to execution by a server, causes the server to retrieve healthcare information from one or more databases, generate a plurality of drug monographs using the healthcare information retrieved from the one or more databases and based on user input from a plurality of users, monitor activity of the plurality of users during the generation of the plurality of drug monographs, and generate a report indicating a pattern of usage associated with one or more of the plurality of drug monographs based on the monitored activity of the plurality of users. In some embodiments, the pattern of usage may include trend and occurrence information associated with one or more of the plurality of drug monographs. In some embodiments, to monitor the activity of the plurality of users during the generation of the plurality of drug monographs comprises to identify a frequency of one or more studies being included, excluded, viewed, or not viewed in preparation of the plurality of drug monographs. In some embodiments, the one or more studies may include one or more client or competitor studies. In some embodiments, to monitor the activity of the plurality of users during the generation of the plurality of drug monographs may include to identify a geographic region associated with each user generating a drug monograph. In some embodiments, to generate the report indicating the pattern of usage associated with one or more of the plurality of drug monographs may include to indicate a pattern of usage associated with the healthcare information.

Further embodiments, forms, features, and/or aspects of the present application shall become apparent from the description and figures provided herewith.

### BRIEF DESCRIPTION OF THE DRAWINGS

The concepts described herein are illustrative by way of example and not by way of limitation in the accompanying figures. For simplicity and clarity of illustration, elements illustrated in the figures are not necessarily drawn to scale. Where considered appropriate, references labels have been repeated among the figures to indicate corresponding or analogous elements.

FIG. 1 is a block diagram of at least one embodiment of a system for facilitating the generation of one or more drug monographs;

FIG. 2 is a task selection screen configured to enable a user to create and view monographs and to manage a user account.

FIGS. 3-16 are user interface screens accessible to a user to generate a monograph with publically available information and proprietary information;

FIG. 17 is a simplified flow diagram of at least one embodiment of a method for generating a report providing a summary of a database of drug monographs; and

FIG. 18 is a table identifying various example categories determined from the database of drug monographs that may be conveyed to a user in a report.

### DETAILED DESCRIPTION

Although the concepts of the present disclosure are susceptible to various modifications and alternative forms, specific embodiments have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should further be appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

The disclosed embodiments may, in some cases, be implemented in hardware, firmware, software, or a combination thereof. The disclosed embodiments may also be implemented as instructions carried by or stored on one or more transitory or non-transitory machine-readable (e.g., computer-readable) storage media, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may not be included or may be combined with other features.

As described in detail below, in the illustrative embodiment, the system 100 generates and maintains a subscription-based, efficient, and user-friendly interface to facilitate and expedite the generation of drug monographs including health system drug class reviews. The system 100 is intended for use by practitioners, and more particularly to clinical pharmacists and others who support or have an interest in the formulary decision processes at United States health plans, integrated delivery networks, hospitals, and medical groups. The users of the system include, but are not limited to payer pharmacists, formulary decision makers (such as integrated delivery networks (IDNs), accountable care organizations (ACOs), hospitals, pharmaceutical manufacturers (Managed Markets, Marketing, Medical), and pharmacy schools/students. The system 100 provides a user experience that is simple, fast, and intuitive and substantially reduces the amount of time expended on monograph creation. It should be appreciated that a drug monograph may discuss, among other characteristics and parameters, the clinical advantages and disadvantages of particular drug for use by a particular institution.

The system 100 facilitates the generation of drug monographs/formularies though the use of an electronic user interface, a graphical user interface (GUI) accessible (e.g., through a web browser) to access a website having one or more databases. The user interface provides a variety of functions including guiding the user through a series of steps to ultimately deliver a drug class review customized to his/her professional judgment. The user interface enables a user to search designated external websites for published research studies which can be reviewed and selected for inclusion in the user's drug class review. Once published research is selected by the user, information automatically populates the selected studies into a bibliography for a given review. In the illustrative embodiment, all studies reviewed and the selected studies are time stamped with dates identifying the date of access and the web address (or other access location) of the location of the studies. The system 100 provides significant analytical capabilities to track each user's interaction with the system including, for example, documents reviewed and patterns of reviews made by the user. The patterns or behaviors may be stored and analyzed, and reports may be generated to include a broad range of behavior patterns and preferences of the user community or third parties. Reports can be made available to one or more users of the system 100 offering aggregated trend information about the reviews being conducted on the site. Depending on the particular embodiment, such reports may be made available on a periodic basis (e.g., quarterly or monthly) or according to another suitable schedule (e.g., on demand).

Each user may be provided with the option of a subscription service for predetermined periods of time. For instance, in some embodiments, subscribed users are notified of new prescribing instructions and newly published studies which are relevant to the user's recent searches. File storage may be provided for each user or for each account for future retrieval of relevant data. In the illustrative embodiment, it should be appreciated that the system maintains the proprietary nature of the drug class reviews created by each subscriber, so that each subscriber only has access to the reviews that the user or other users within the same organization has prepared. The system may also enable users to opt-in to future system enhancements.

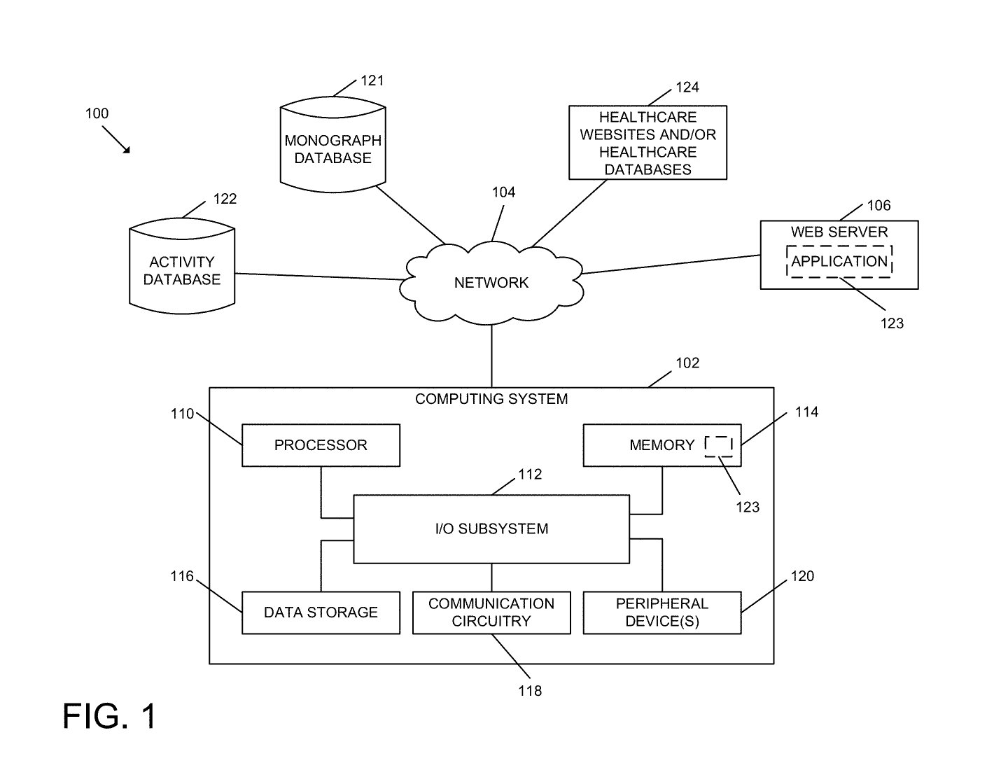

Referring now to FIG. 1, an illustrative system 100 for facilitating the generation and distribution of a drug monograph includes a computing device 102, a network 104, and a web server 106. The computing device 102 may be embodied as any type of computing device capable of performing the functions described herein. For example, the computing device 102 may be embodied as a desktop computer, laptop computer, tablet computer, notebook, netbook, Ultrabook™, cellular phone, smartphone, wearable computing device, personal digital assistant, mobile Internet device, Internet of Things (IoT) device, server, router, switch, and/or any other computing/communication device capable of performing the functions described herein. As shown in FIG. 1, the illustrative computing device 102 includes a processor 110, an input/output (“I/O”) subsystem 112, a memory 114, data storage 116, a communication circuitry 118, and one or more peripheral devices 120. The computing device 102 may include other or additional components, such as those commonly found in a typical computing device (e.g., various input/output devices and/or other components), in other embodiments. Additionally, in some embodiments, one or more of the illustrative components may be incorporated in, or a portion thereof, may be incorporated in the processor 110 in some embodiments. Although a single computing device 102 is illustratively shown, it should be appreciated that one or more of the components of the computing device 102 described herein may be distributed across multiple computing devices. In other words, the techniques described herein may be employed by a computing system that includes one or more computing devices.

The processor 110 may be embodied as any type of processor capable of performing the functions described herein. For example, the processor 110 may be embodied as a single or multi-core processor(s), digital signal processor, microcontroller, or other processor or processing/controlling circuit. Similarly, the memory 114 may be embodied as any type of volatile or non-volatile memory or data storage capable of performing the functions described herein. In operation, the memory 114 may store various data and software used during operation of the computing device 102 such as operating systems, applications, programs, libraries, and drivers. The memory 114 is communicatively coupled to the processor 110 via the I/O subsystem 112, which may be embodied as circuitry and/or components to facilitate input/output operations with the processor 110, the memory 114, and other components of the computing device 102. For example, the I/O subsystem 112 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 112 may form a portion of a system-on-a-chip (SoC) and be incorporated, along with the processor 110, the memory 114, and other components of the computing device 102, on a single integrated circuit chip. For example, in some embodiments, one or more of the components of the computing device 102 may form one or more application-specific integrated circuits (ASICs).

The data storage 116 may be embodied as any type of device or devices configured for short-term or long-term storage of data such as, for example, memory devices and circuits, memory cards, hard disk drives, solid-state drives, or other data storage devices. The data storage 116 and/or the memory 114 may store various data during operation of the computing device 102 useful for performing the functions described herein.

The communication circuitry 118 may be embodied as any communication circuit, device, or collection thereof, capable of enabling communications between the computing device 102 and other remote devices (e.g., the web server 106) over a network (e.g., the network 104). The communication circuitry 118 may be configured to use any one or more communication technologies (e.g., wireless or wired communications) and associated protocols (e.g., Ethernet, Bluetooth®, WiMAX, etc.) to effect such communication.

The peripheral devices 120 may include any number of additional peripheral or interface devices, such as speakers, keyboards, microphones, additional storage devices, and so forth. The particular devices included in the peripheral devices 120 may depend on, for example, the type and/or intended use of the computing device 102. For example, in various embodiments, the peripheral devices 120 include a keyboard, mouse, display, touchscreen display, printer, alarm, status indicator, handheld device, diagnostic tool, reader device, and/or one or more other suitable peripheral devices 120.

The network 104 may be embodied as any type of communication network capable of facilitating communication between the computing device 102 and remote devices (e.g., the web server 106). As such, the network 104 may include one or more networks, routers, switches, computers, and/or other intervening devices. For example, the network 104 may be embodied as or otherwise include one or more cellular networks, telephone networks, local or wide area networks, publicly available global networks (e.g., the Internet), ad hoc networks, short-range communication links, or a combination thereof.

The web server 106 may be embodied as any type of computing device capable of performing the functions described herein. For example, the web server 106 may be embodied as a server, desktop computer, laptop computer, tablet computer, notebook, netbook, Ultrabook™ cellular phone, smartphone, wearable computing device, personal digital assistant, mobile Internet device, Internet of Things (IoT) device, router, switch, and/or any other computing/communication device capable of performing the functions described herein. In some embodiments, the web server 106 may be similar to the computing device 102 described above. For example, the web server 106 may include components similar to the components of the computing device 102 described above and, therefore, the descriptions of those components have not been repeated herein for simplicity of the description. Further, it should be appreciated that the server 106 may include other components, sub-components, and/or devices commonly found in a computing device, which are also not discussed herein for simplicity of the description. Additionally, in some embodiments, one or more of the components of the computing device 102 may be omitted from the server 106 (e.g., the peripheral devices 120).

Although only one computing device 102, one network 104, and one web server 106 are shown in the illustrative embodiment of FIG. 1, the system 100 includes, in different embodiments, multiple computing devices 102, multiple networks 104, and/or multiple servers. For example, in some embodiments, a single computing device 102 communicates with multiple servers 106.

It should be further appreciated that, although the web server 106 is described herein as a computing device outside of a cloud computing environment, in other embodiments, the server 106 may be embodied as a cloud-based device or collection of devices within a cloud computing environment. Further, in cloud-based embodiments, the server 106 may be embodied as a “serverless” or server-ambiguous computing solution, for example, that executes a plurality of instructions on-demand, contains logic to execute instructions only when prompted by a particular activity/trigger, and does not consume computing resources when not in use. That is, the server 106 may be embodied as a virtual computing environment residing “on” a computing system (e.g., a distributed network of devices) in which various virtual functions (e.g., Lamba functions, Azure functions, Google cloud functions, and/or other suitable virtual functions) may be executed corresponding with the functions of the server 106 described herein. For example, when an event occurs (e.g., data is transferred to the server 106 for handling), the virtual computing environment may be communicated with (e.g., via a request to an API of the virtual computing environment), whereby the API may route the request to the correct virtual function (e.g., a particular server-ambiguous computing resource) based on a set of rules. As such, when a request for the transmission of certain data is made (e.g., via an appropriate user interface to the server 106), the appropriate virtual function(s) may be executed to perform the actions before eliminating the instance of the virtual function(s).

It should be appreciated that the computing device 102 may be configured to execute an application 123 that is stored locally and/or remotely relative to the computing device 102. Further, the application 123 may be embodied as any type of application suitable for performing the functions described herein. In particular, in some embodiments, the application 123 may be embodied as a web application, a mobile application (e.g., smartphone application), a cloud-based application, a thin-client application, and/or another type of application. Although depicted as an application of the computing device 102, in some embodiments, at least a portion of the application 123 may be executed by the web server 106. For example, in some embodiments, the application 123 of the computing device 102 may serve as a client-side interface (e.g., via a web browser) for a web-based application or service (e.g., of the web server 106). Although the application 123 is primarily described herein as an interface to a web-based application accessible via the web server 106, it should be appreciated that the technologies described herein apply equally well to embodiments in which the application 123 is a smartphone application and/or other type of application. In some embodiments, a user (e.g., a stakeholder) of the system 100, communicates using and/or interacting with the web server 106 through a graphical user interface (GUI) of the application 123 (e.g., a web-based application accessible via the web server 106). The web server 106 is configured to enable the user to facilitate and expedite the generation of drug monographs/formularies including health system drug class reviews. For simplicity of the description, it should be appreciated that the application 123 may be described herein as being an application of the computing device 102 or an application of the web server 106; however, unless expressly stated to the contrary, the corresponding description is not intended to be so limiting.

In the illustrative embodiment, the network 104 is further communicatively coupled to one or more healthcare websites and/or one or more healthcare databases 124. In particular, in the illustrative embodiment, the websites/databases 124 may be accessible using the web server 106, which may retrieve information requested by a user via the application 123 of the computing device 102. In some embodiments, the websites/databases 124 include healthcare information directed to drug information including acceptable ingredients, dosing, formulation, labeling, safety, effectiveness, active ingredients, and/or other relevant drug information. Such websites and/or databases may include, for example, those maintained by the National Institute of Health (NIH), the Logical Observation Identifiers Names and Codes Organization (LOINC.org), the US National Library of Medicine National Institutes of Health, the Managed Markets Insight & Technology (MMIT) network, and/or the Food and Drug Administration (FDA). In some embodiments, the database information is accessed using an application program interface (API). The section below titled “APIs for Access to Healthcare Websites and Databases” provides a description of various APIs that may be included to access the information provided by those databases.

In some embodiments, the application 123 may serve as a client-side interface to a web-accessible, software-as-a-service application (e.g., served by the web server 106) designed to provide stakeholders in formulary decision processes with a highly-efficient, scalable, usable, and distributable monograph- and formulary-creation product by amalgamating disparate information from several sources into a single interface.

In some embodiments, the application 123 may include user access control, external support/contact pages, cron-based or action-based email automation, monograph editing, review, distribution, archival, subscription access, customer and user information management, reports, a demonstration mode, and/or administrative panels to manage these functions. In some embodiments, exhaustive user activity reports may be generated (e.g., periodically and/or on demand) including psychographic, demographic, and behavioral analysis reports. In some embodiments, a full suite of Google Analytics is used to generate a report. Google Analytics reference codes may be used. In some embodiments, the application 123 may be configured to create custom reports for administrative review including, for example, reports describing industry benchmarks, manufacturer study details, and/or other types of reports.

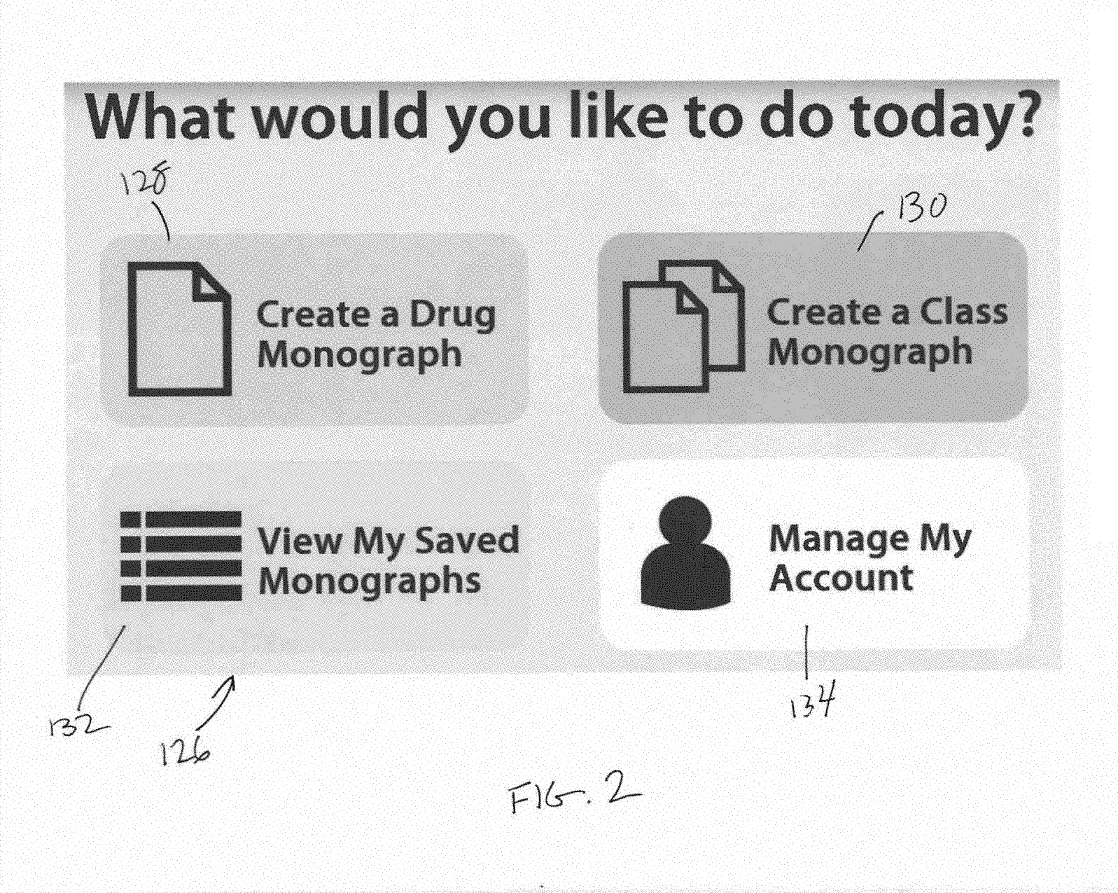

The illustrative application 123 includes a login screen, which may be used as an access portal for users. Once a user is logged in, the user is presented with a task selection screen 126 as illustrated in FIG. 2. As shown, the illustrative task selection screen 126 includes one or more selectable user interface elements including a Create a Drug Monograph button 128, a Create a Class Monograph button 130, a View Saved Monographs button 132, and a Manage My Account button 134. Each of the screens, as described herein, are presented on the GUI and may include a unique configuration.

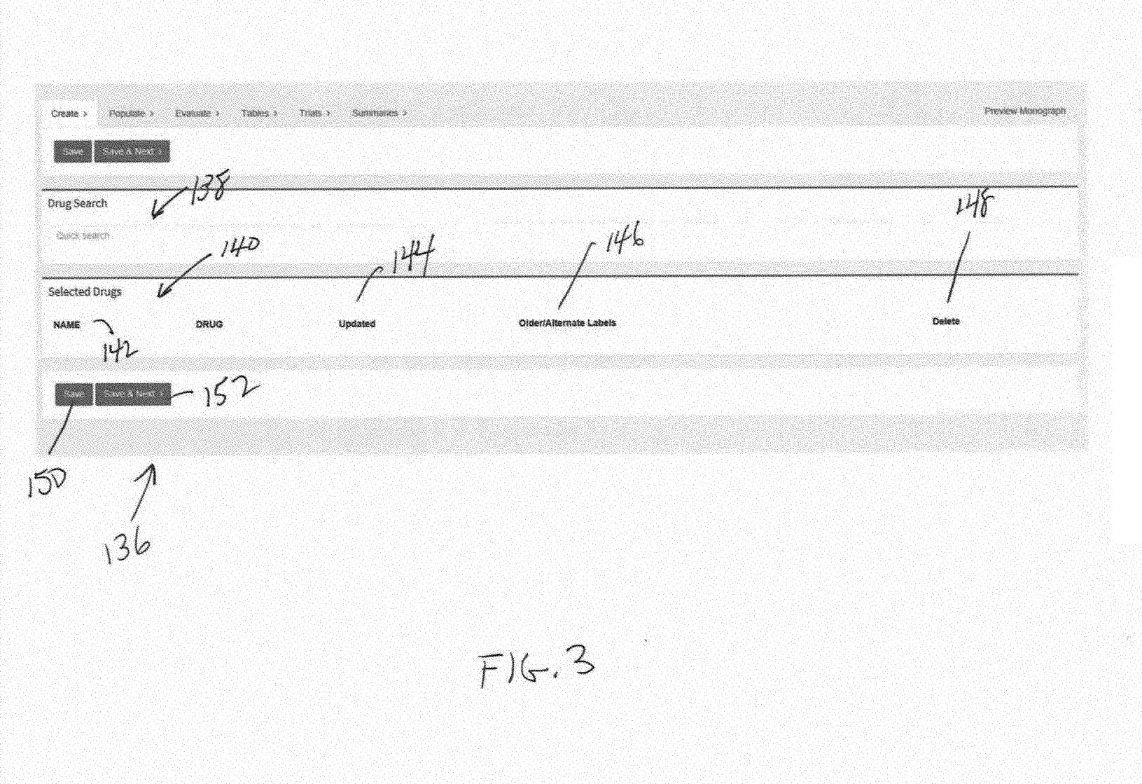

Upon selection of the Create Drug Monograph button 128 as presented by the task selection screen 126, the user is presented with a create screen 136 as illustrated in FIG. 3, which provides for the preparation and editing of a drug monograph. In the illustrative embodiment, the create screen 136 provides a search bar 138, which accepts user input provided to the computing device 102 (e.g., via a peripheral device, such as a keyboard, touchscreen, audio, and/or other I/O device). Upon entering the name of a drug into the search bar, for instance, one or more drug names populate a selected drugs table 140. The illustrative selected drugs table 140 identifies each of the drugs returned by the search by the name 142 of the drug, an update 144 identifying the date when the drug information was updated, and (if any) an older or alternate label 146 of the drug. Each entry further includes a delete selector 148 that enables the user to delete one or more of the drugs provide by and listed in the selected drugs table 140.

When a search is made, the number of drugs returned by the selected drugs search can exceed one. As such, in the illustrative embodiment, the user is given the option to delete one or more of the drugs from the list by using the delete selector 148. Once a drug of interest is selected by deleting those drugs not needed, the selected drug is saved (e.g., by the web server 106) by selecting a Save button 150 or a Save and next button 152. In other embodiments, it should be appreciated that the drug(s) of interest may be otherwise selected/filtered (e.g., by affirmative selection rather than deletion).

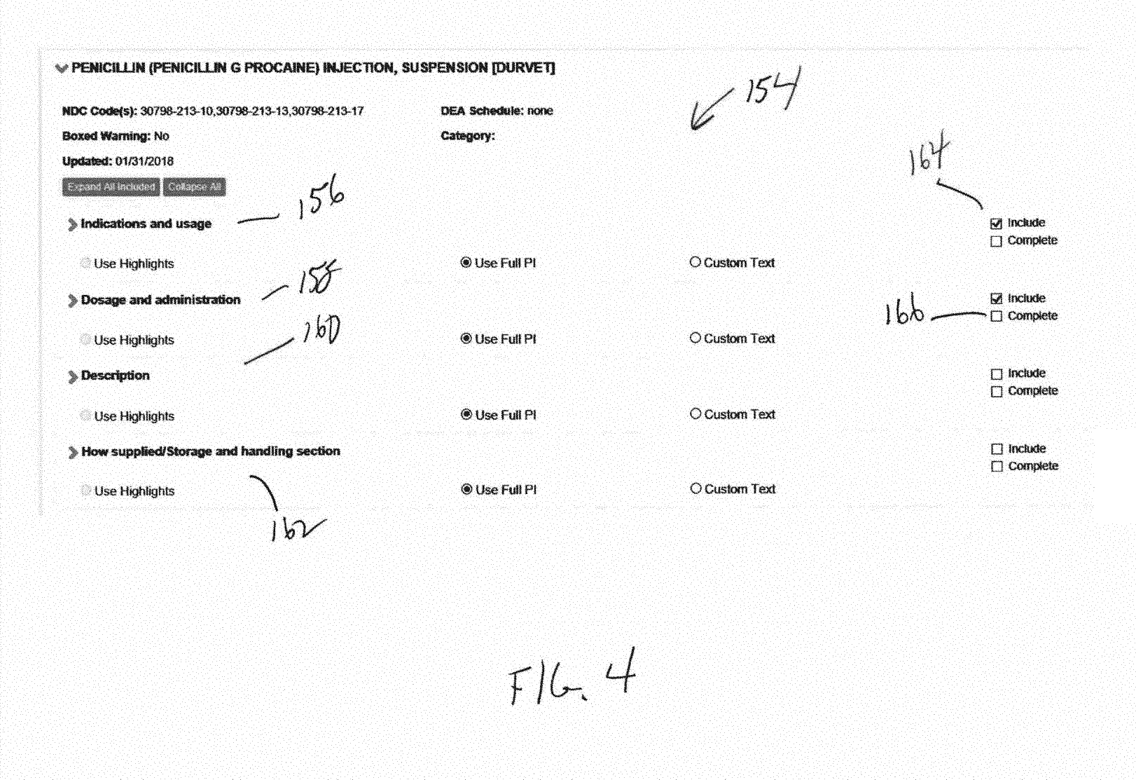

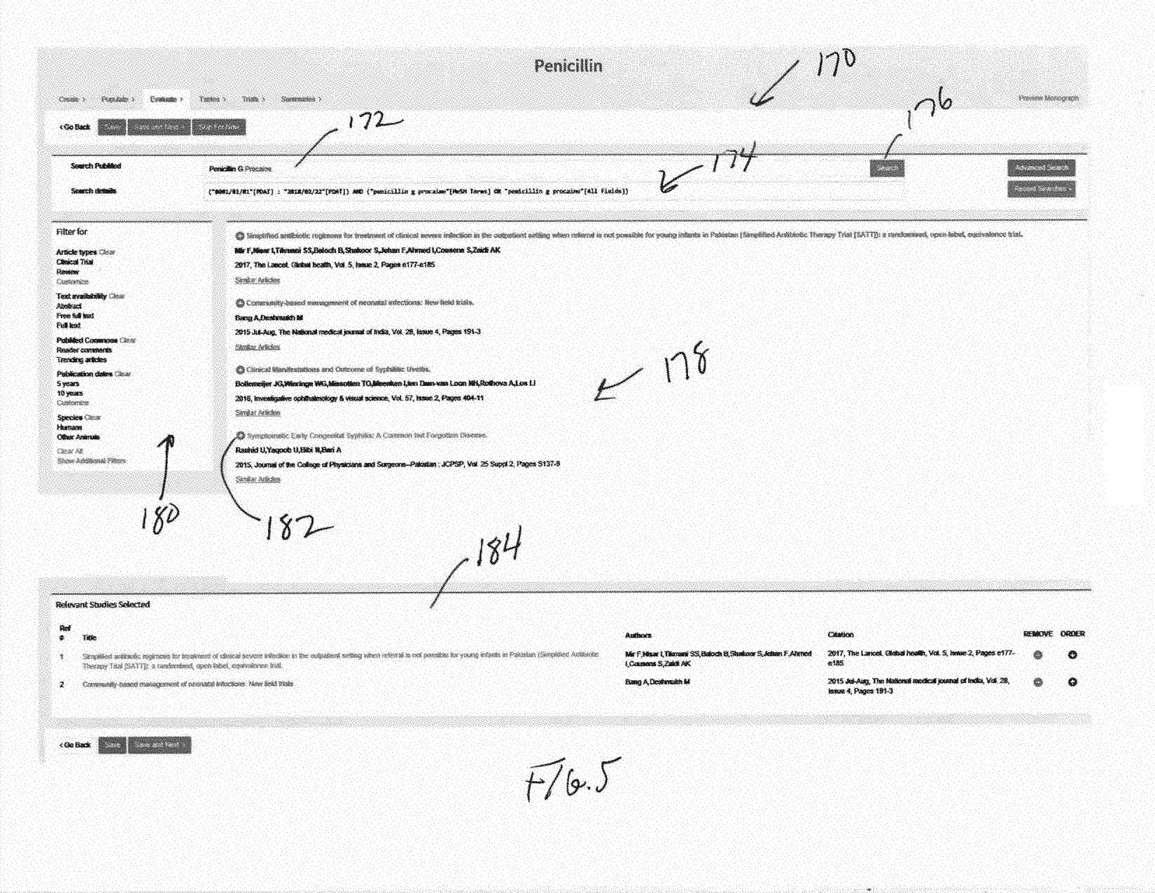

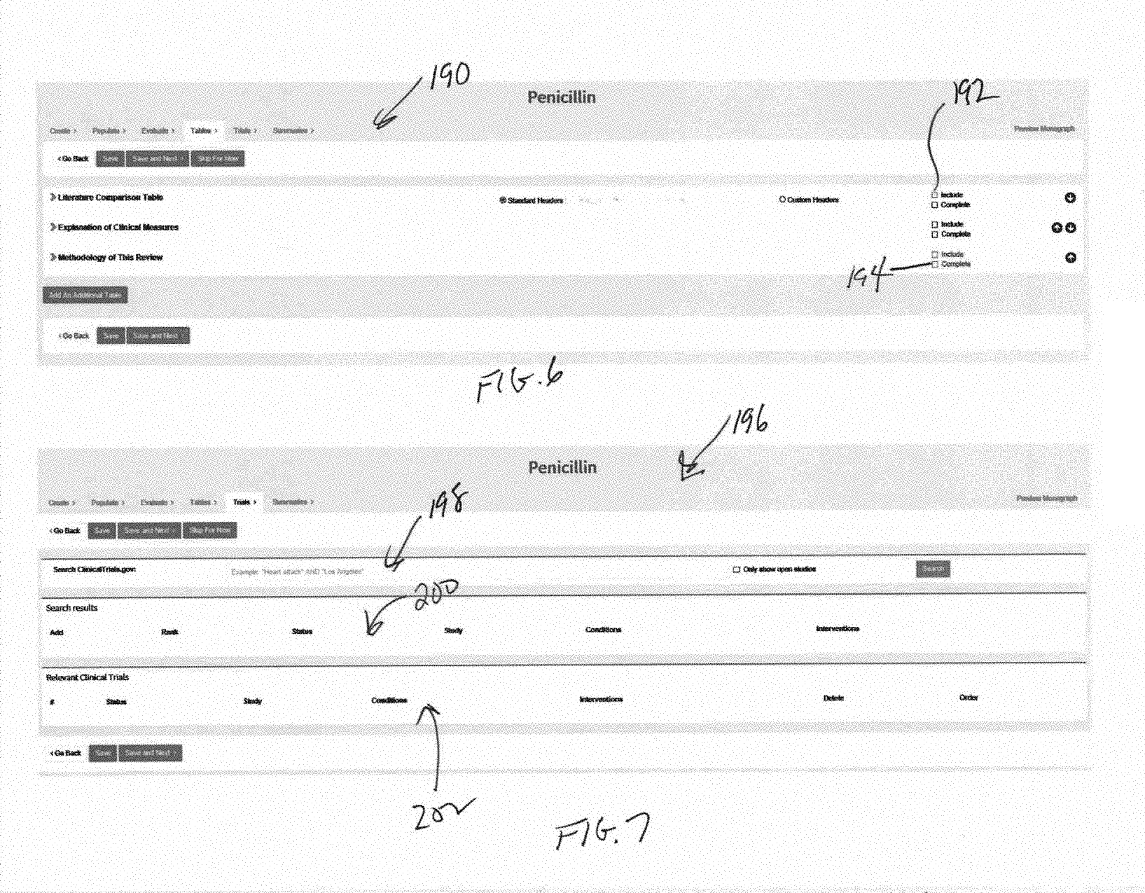

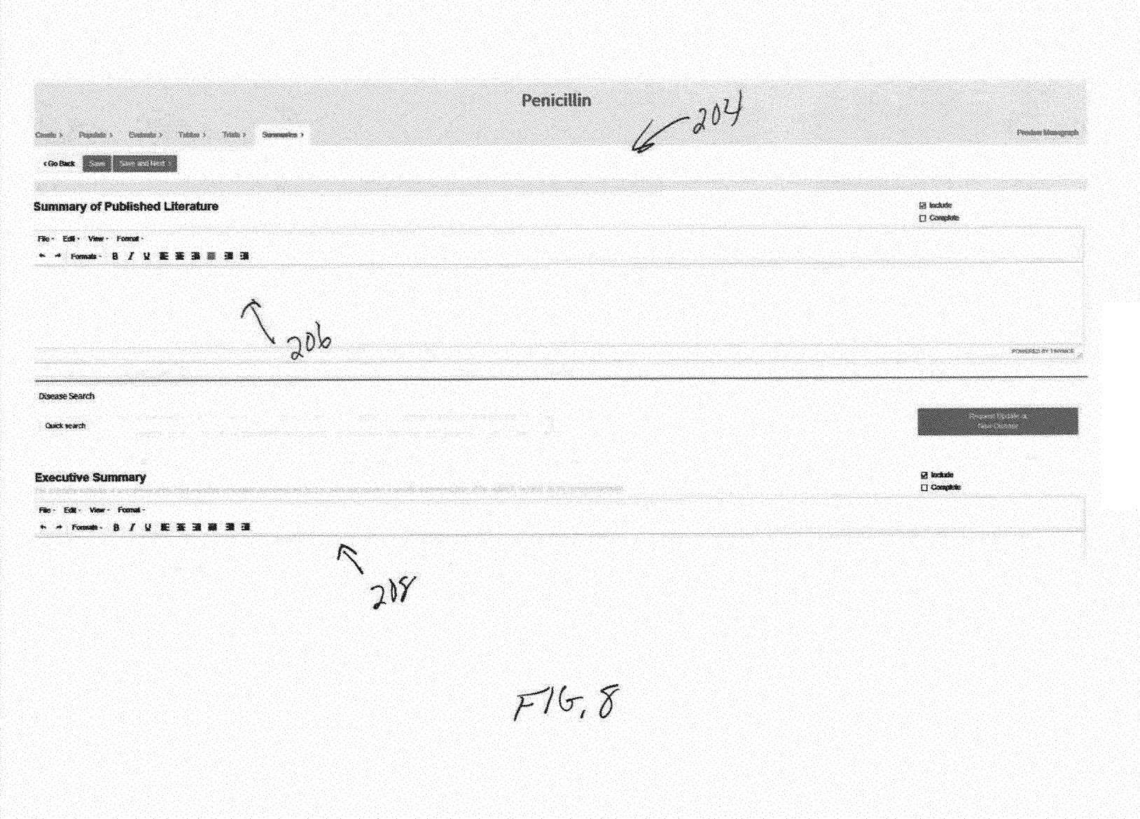

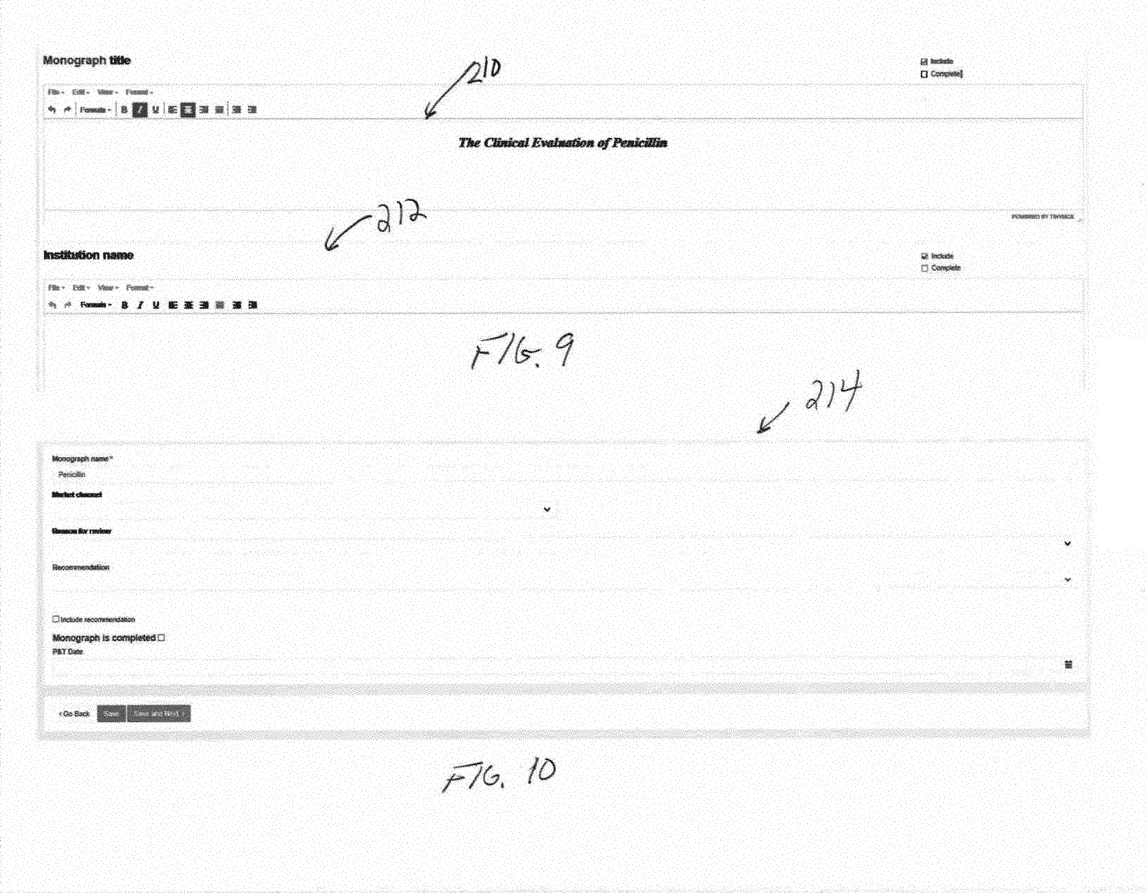

FIG. 4 illustrates an information screen 154 for the drug Penicillin identified in FIG. 3, and selecting a particular result of Penicillin (Penicillin G Procaine) Injection, Suspension provided by the company Durvet. The illustrated information includes one or more national drug codes (NDC), the Drug Enforcement Agency (DEA) Schedule, the existence of a boxed warning, a category, and a date at which the information was updated. Additional information is also provided upon selection of the type of drugs and the user has the ability to gather and organize the information based on a number of selection tabs and/or other user interface elements. In particular, in the illustrative embodiment, the information screen 154 includes tabs entitled indications and usage tab 156, dosage and administration tab 158, description tab 160, and how supplied/storage and handling section tab 162. In the illustrative embodiment, each of these tabs enables the user to select from one of a plurality of different types of information including: use highlights, use full prescribing information (PI), and custom text. The type of information to be included in the final monograph is initially saved by checking an include box 164. Once the user has made a final decision, checking a complete box 166 may indicate that the monograph has been reviewed and no longer needs to be edited.

Once the desired information has been selected, a publication screen 170, as illustrated in FIG. 5, is presented to the user. In this screen, the user may search, for example, the PubMed citations maintained by the National Library of Medicine. In the illustrative embodiment, the drug, Penicillin G Procaine, is displayed in a Search PubMed text box 172, which is automatically populated by the application 123. In another embodiment, the information may be entered manually by the user. A search details text box 174 is populated with one or more search terms generated by the application 123. Upon selection of a Search button 176 by the user, search results are provided in a search results box 178. In the illustrative embodiment, four documents are provided, each of which can be included as a document of a citation in the final monograph. A filter box 180 is provided for a user to filter the results of the search. Depending on the particular embodiment, the results can be limited or filtered before the search is performed and/or after the results have been provided. Once the user has determined which of the publications is/are appropriate for the monograph, the selected ones are added to the monograph by selecting a plus sign 182 (or other suitable user interface element). In the illustrative embodiment, the studies selected are shown in a relevant studies select text box 184. Title, authors, and citation are shown in at least one embodiment. Additional and/or alternative information may be shown in other embodiments.

Once the evaluation has been completed, the user organizes the information in tables at a tables user interface 190 illustrated in FIG. 6. The illustrative table includes one or more standard headers provided by the application 123 (e.g., a web-based application). In some embodiments, the tables include a literature comparison table, an explanation of clinical measures table, and a methodology of review table. As with other aspects of the graphical user interface and the monograph creation system described above, in the illustrative embodiment, the user is given the opportunity to include each of the selections with an include box 192 and upon making a determination of the value of the table, the user completes the selection by selecting the complete box 194.

After generating the relevant tables, the user is given the option to include trials in a trials user interface 196 as illustrated in FIG. 7. In the illustrative embodiment, a search tab 198 includes a user-fillable text box where the user is able to input one or more keywords to search for clinical trials at the U.S. Government Clinical Trials website and/or other websites/databases that store searchable data associated with clinical trials. The search results are shown in a search results box 200, which may be selected by the user if considered sufficiently relevant to the monograph via a relevant clinical trials box 202. Once completed, the user saves the results with a Save button or a Save and Next button.

Upon completion of the trials user interface, in the illustrative embodiment, the user is presented with a summaries user interface 204 as illustrated in FIGS. 8 and 9. As shown in FIG. 8, the summaries user interface 204 includes a text box 206 for the user to summarize the published literature and a text box 208 for the user to provide an executive summary. In some embodiments, the user provides the information for each of the text boxes 206, 208. In other embodiments, the application 123 populates each of the text boxes 206, 208 with initial information (e.g., stock language), which is editable by the user.

In some embodiments, when the user clicks the text link in the “Executive Summary” column, a light box pop-up will be displayed containing the contents of the “Executive Summary” field, including text and paragraph formatting. In some embodiments, clicking the “Share” icon may hide the “Share” icon and display a set of three icons: “Print”, “Link”, and “Email.” In some embodiments, clicking the “Print” icon will either display a PDF of the monograph or download it to the user's computer (e.g., based on the user's current browser or application 123 settings).

Clicking the “Link” icon may display a light box dialog with two options: create a link that is valid up through a certain date, or create a link that expires when the monograph is archived, expires, or is re-opened for review. Once selected, the user can click the “Create a Link to this Monograph” button. In some embodiments, doing so will display a globally unique identifier (GUID) created and a button labeled “Copy the Link to this Monograph”. Further, a message may be displayed indicating that the link has been copied to the clipboard. The user may also click the “Cancel” link to close this window in the “Copy” state or the “Create” state of the dialog.

Once created, the GUID may stored in a database (e.g., accessible by the web server 106), making it accessible by and/or for the user so that the user does not need to separately store or remember the GUID outside the system 100. In order to recall a link, the user may click on the “Share” icon, choose the “Link” icon, and instead of creating a new link, click the “View Existing Links” button. A list of all active GUID links may be displayed, including the date after which the link expires. The user can select a link and click the “Copy the Selected Link.” In some embodiments, doing so may display a message that the link has been copied to the clipboard. The user may also click the “Cancel” link to close this state of the dialog or go back to the “Create” state and create a new link by clicking the “Create a New Link” button.

In another embodiment, clicking the “Email” icon may open a light box dialog explaining to the user how to email one or more users. The user can cancel this dialog by clicking the “Cancel” text link if they choose. Or, the user may enter a value in the “Email” field and click the “Share via Email” button. Once clicked, the dialog may present a confirmation, reveal the View Monographs screen again, generate a GUID as described herein, and send an email with the PDF attachment to all users entered.

In still another embodiment, there may be a two-step process for sharing via email. In particular, when the user clicks the “Email” icon for the first time, a confirmation dialog may appear asking the user to confirm his or her email address. The user can cancel this dialog by clicking the “Cancel” text link, if they choose. Or, the user can enter or edit the value in the “Email” field (e.g., pre-populated from [user email]) and click the “Confirm” button. Once clicked, the dialog may present a confirmation, reveal the View Monographs screen again, generate a GUID as described herein, and send a confirmation email with the GUID to the user.

In the illustrative embodiment, the GUID may do several things when clicked including marking the user's email as confirmed, linking the now-confirmed user to the View Monographs screen, and displaying a “Share via Email” pop-up. In some embodiments, the user may be automatically navigated to this screen (e.g., regardless of whether the user has to login).

Once confirmed, the “Share via Email” light box dialog may display an email address (e.g., as a read-only label) where the normal “Email” field is such that it cannot be edited or changed. Clicking the “Share via Email” button may display a confirmation message to the user and send the generated PDF to the user. Further, clicking the “Edit” icon may take the user to the monograph editor, and clicking the “View” icon may display the monograph in PDF form. A monograph title text box 210 may be displayed for the user to provide a title of the monograph. An institution name title box 212 may be displayed for the user to provide the name of the institution generating and publishing the monograph.

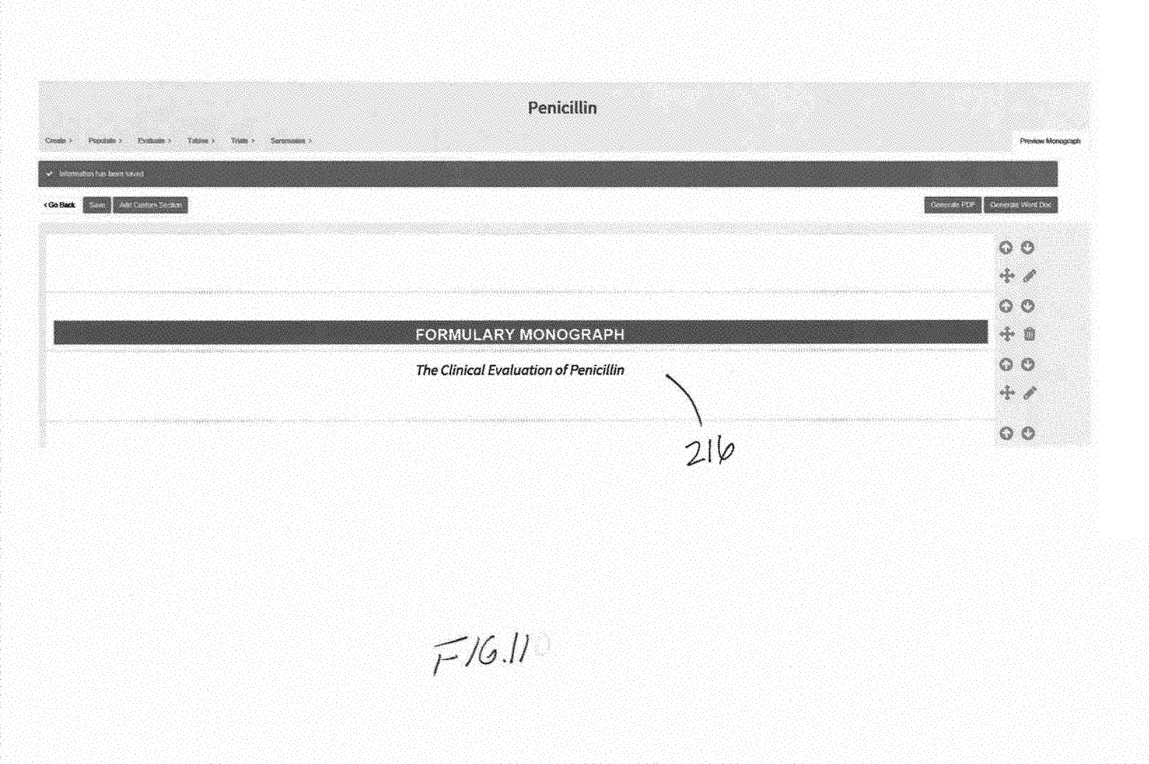

FIG. 10 illustrates a user review screen 214 providing the user with a summary of the created monograph including a monograph name, a market channel, a reason for the review, and a recommendation. Once reviewed by the user, the user makes a determination of whether the completed monograph is acceptable to the organization. In some embodiments, the recommendation can be included by checking the appropriate box. Once reviewed and approved, the user checks the monograph completed box. The section below titled “Monograph Editor” includes additional details describing at least one embodiment of the monograph editor described and/or referenced herein.

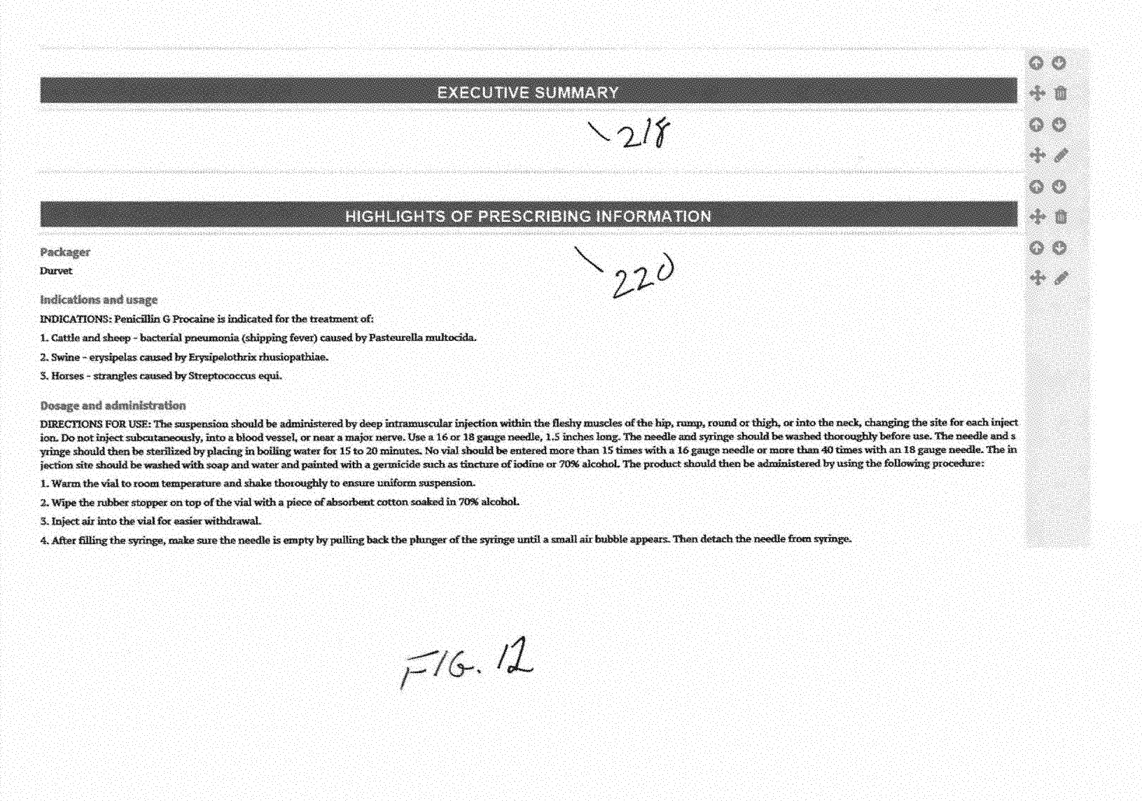

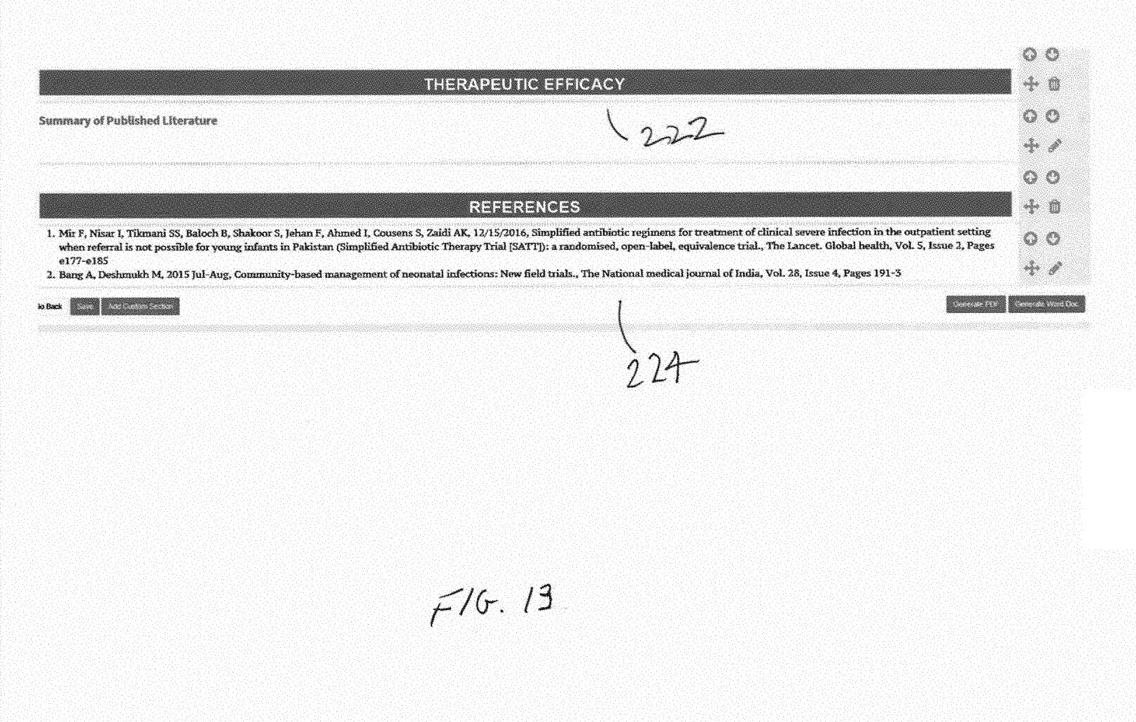

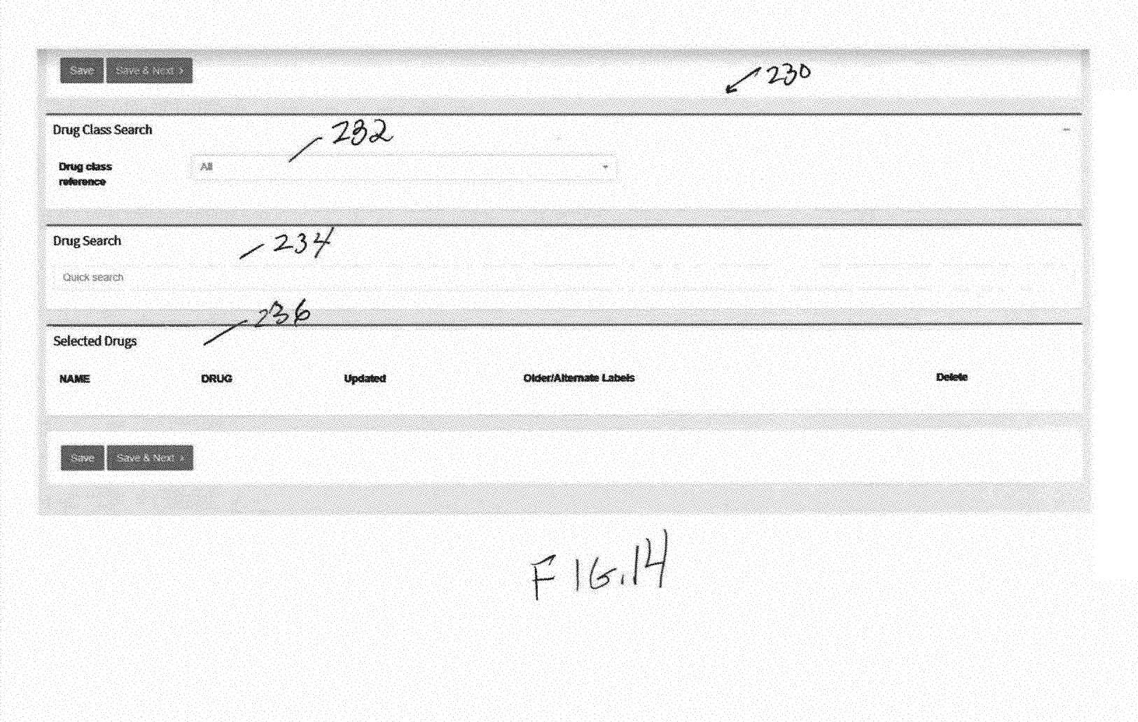

FIGS. 11-13 illustrate a completed monograph including a title 216 (“The Clinical Evaluation of Penicillin” in this embodiment) as shown in FIG. 11, an executive summary 218 and highlights of prescribing information 220 as shown in FIG. 12, and a therapeutic efficacy 222 and references 224 as shown in FIG. 13. It should be appreciated that the completed monograph may be presented to a user for review. In simple terms, the monograph review may display a read-only view of each included section from the monograph editor. Further, in some embodiments, an “Edit” icon to the right of each section may take the user to the applicable tab in the monograph editor when clicked. “Up arrows” and “down arrows” under the “Edit” icon for each section permit the user to scroll up or down through that section. A “Generate PDF” button and a “Generate Word Doc” button may be provided (e.g., at the top of the user interface). Using a utility such as PHPWord or another suitable utility (if necessary), clicking the “Generate Word Doc” button may prompt the user to download a Word document when clicked. The same formatting rules, including which sections and section headers are shown or hidden, may apply to the Word document so that the resultant Word and PDF files appear the same when opened.

In some embodiments, the “Bibliography” section may appear on the monograph review screen but not in the editor. True to form for a bibliography, a reference number may be stored in numerical order of the rows in the Relevant Studies Selected table. In the illustrative embodiment, the bibliography itself is at the end of the monograph review and cannot be moved or edited. Each row in the bibliography may be preceded with the corresponding citation number from the Relevant Studies Selected table.

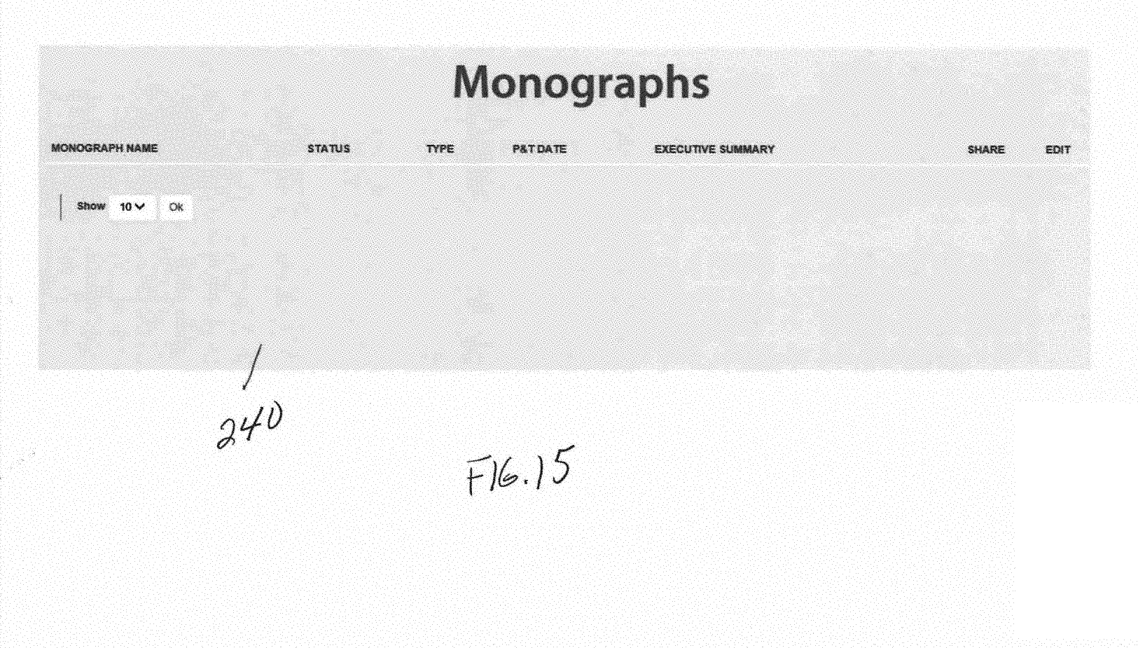

Referring back to FIG. 2, in addition to generating a drug monograph using the Create a Drug Monograph button 128, the task selection screen 126 also provides the user with an option to generate a class monograph by selecting the Create a Class Monograph button 130. After selection of the Create a Class Monograph button 130, in the illustrative embodiment, the user is presented with a drug class search screen 230 of FIG. 14. A drug class reference text box 232 provides a location for the user to input drug class information to be searched. The illustrative drug class screen 230 includes a drug search text box 234, which accepts user input providing a particular drug to be searched. The results of the search may be displayed in a selected drugs list 236, which may list the results of the search by name, drug, date of an update of the information retrieved, and older or alternate labels. The user may also be given the option of deleting one or more of the listed drugs using a Delete button similar to that described above.

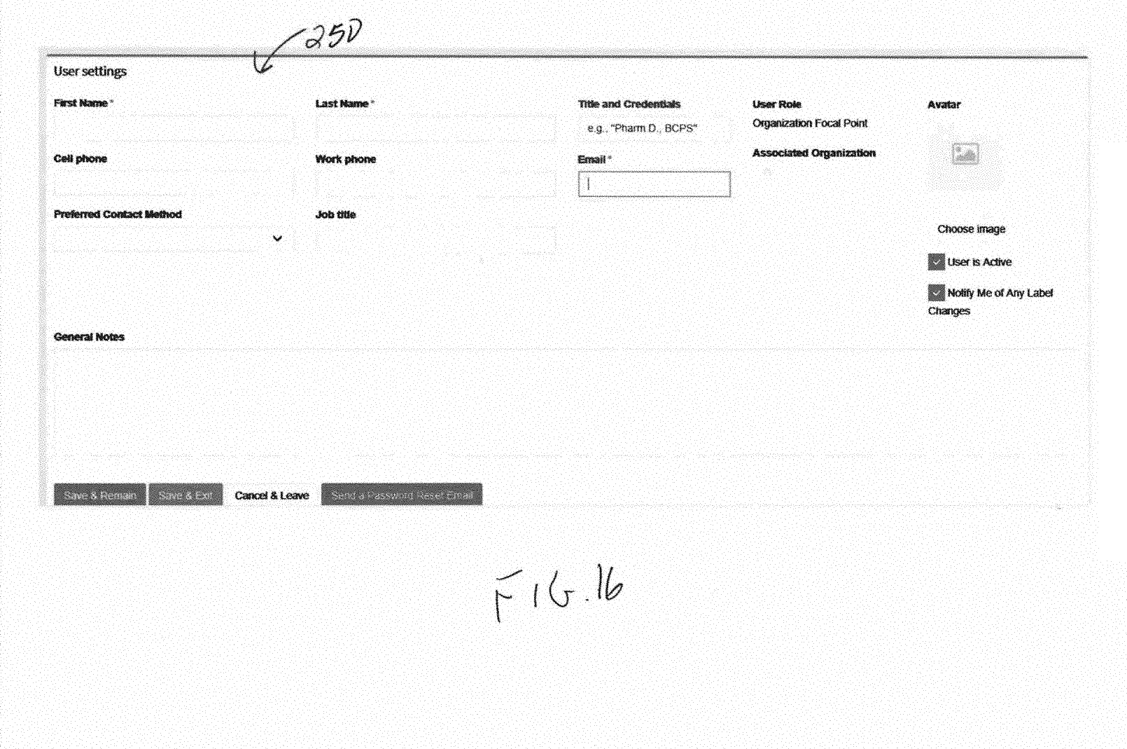

Referring back to FIG. 2, after each of the generated monographs has been completed and saved, in the illustrative embodiment, the user is given the option to view the saved monographs with the View My Saved Monographs button 132. Upon selection of this button 132, a list of saved monographs is presented to the user via a monographs screen 240 of FIG. 15. In the illustrative embodiment, each of the saved monographs is listed in the monographs screen 240 and identifies the monograph name, status, type (drug or class), P&T date, and an executive summary. Further, each of the listed monographs may also identify the individuals and/or organizations that have been authorized to view the information with a share column. The screen 240 further includes an edit column, which enables a user to edit the information within a particular monograph.

In some embodiments, the monographs screen 240 may present a table of archived and incomplete monographs and pagination controls. In some embodiments, the table may include a monograph title, a status indicator, a type indictor (e.g., product, class, etc.), an archive date, an executive summary, an archive indicator, and/or other relevant data. In some embodiments, an administrator may transmit to a user a custom link associated with a new drug for which a monograph is likely to be prepared, which may link the user to a pre-populated (e.g., or partially populated) monograph without the need to search for the drug. In other words, in some embodiments, a portion of the flow for creating a monograph may be bypassed.

In some embodiments, the user may further access a user settings screen 250 as illustrated in FIG. 16. In particular, referring back to the illustrative embodiment of FIG. 2, the user may select the Manage My Account button 134 depicted in FIG. 2 to access the user setting screen 250 of FIG. 16. The illustrative user settings screen 250 includes text boxes which are populated by the user to identify the user including name, contact information, title and credentials, and general notes.

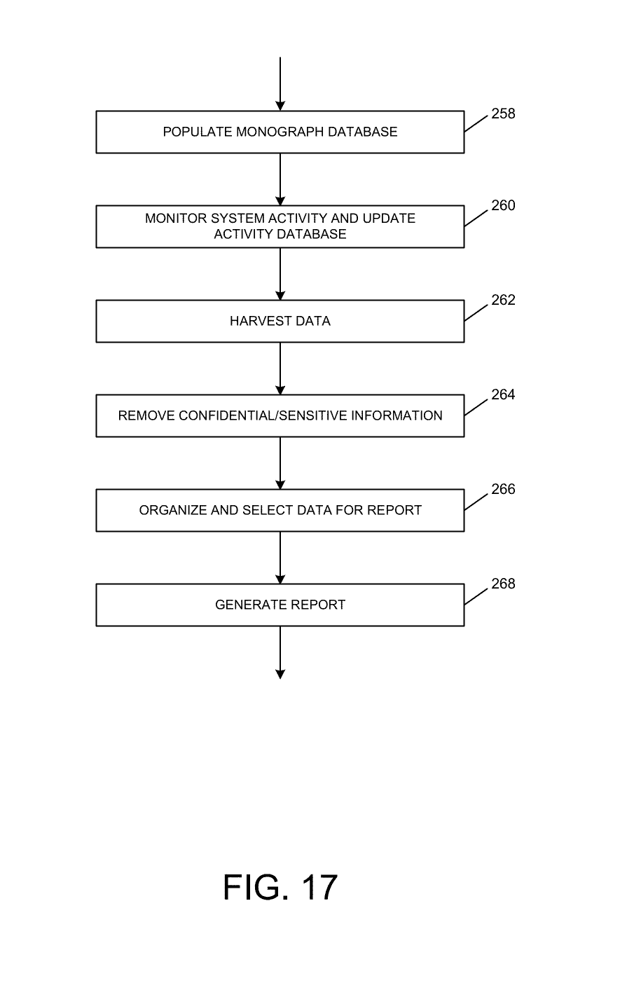

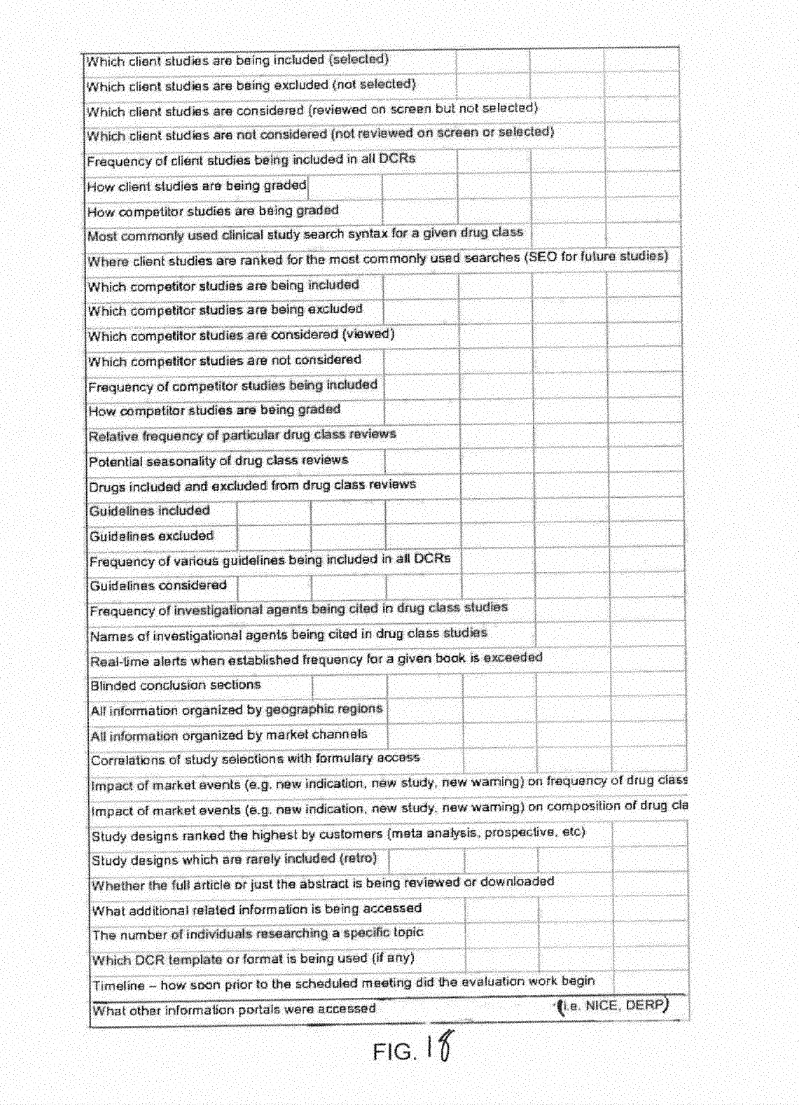

Referring now to FIG. 17, in use, the system 100 may execute a method for generating healthcare information based on one or more drug monographs. It should be appreciated that the particular blocks of the method are illustrated by way of example, and such blocks may be combined or divided, added or removed, and/or reordered in whole or in part depending on the particular embodiment, unless stated to the contrary. The illustrative method begins with block 258 of FIG. 17 in which the system populates the monograph database 121 in a manner consistent with the techniques described herein. For example, in some embodiments, once the completed monographs has been approved, the completed monographs are accessible by the user or client (e.g., a pharmacy) for further use. Since each of the completed monographs includes information that could relevant to the healthcare industry at large, each of the completed monographs is stored in the monograph database 121 accessible by the computing device 102 and/or the web server 106. Although the completed monographs are described herein as being stored in the monograph database 121, it should be appreciated that they may be stored in another suitable storage location in other embodiments.

In block 260, the system monitors for various system activity and populates the activity database 122 based on detected activity. For example, in some embodiments, the system activity may include user requests, user searches, user search results, and/or other data associated with the user's interaction with the system when generating a drug monograph (e.g., whether stored or not stored by a user when generating monographs). It should be appreciated that the system activity information may include data that provides additional insights into the retrieval and use of healthcare information of interest to the third parties. In some embodiments, the system activity information may be anonymized and aggregated to demonstrate trends and unique information in order to provide reliable, competitive market information, comparison data, and other such evidence-based material. As described herein, it should be appreciated that, in some embodiments, the system activity specific data may be extracted, filtered to remove personal identifying information or other confidential information, and provided to users or third parties (e.g., for a fee).

It should be appreciated that the system 100 or, more specifically, the application 123 may capture useful information from each of the monographs and/or the system activity data to improve patient healthcare outcomes (e.g., based on the information stored in the monograph database 121 and/or the activity database 122).

In some embodiments, targeted and organized drug information may be extracted from each of the monographs and be made available to healthcare providers to improve drug usage and delivery. The extracted information is organized according to predefined classes or parameters to indicate patterns of usage by medical facilities and patients. The targeted and organized information may be retrieved from the drug monograph/formulary information and provides an insight into the requested use or actual use by a patient or a medical facility. For instance, the frequency of certain types of drug monographs could indicate a trend in the treatment of certain ailments and diseases. In some cases, the drug monographs/formularies could indicate regions of the country where certain drugs are more often prescribed or requested. This indicator could be instructive to a drug manufacturer to increase production of a certain drug or increase the delivery to a certain region. This indicator could also be used by a healthcare practitioner to determine which disease conditions or ailments occur more often and which require increased monitoring. Additionally, the quantity of a particular research citation selected for inclusion in a drug formulary could indicate the perceived competence of the organization responsible for the cited research.

The completed monographs and system activity data include data that is gathered, weighed, and anonymized to provide baseline information, a valuable resource, for the pharmaceutical and other healthcare industries. As seen in FIG. 18, different types of data may be gathered depending on the particular embodiment, either separately as new data or new data elements stored in the activity database 122, or as existing data as part of completed monographs in the system. For example, the monitored activity may be analyzed to determine which client studies are being included (selected), which client studies are being excluded (not selected), which client studies are considered (reviewed on screen but not selected), which client studies are not considered (not reviewed on screen or selected), the frequency of client studies being included in all DCRs, how client studies are being graded, how competitor studies are being graded, most commonly used clinical study search syntax for a given drug class, where client studies are ranked for the most commonly used searched (SEO for future studies), which competitor studies are being included, which competitor studies are being excluded, which competitor studies are considered (reviewed), which competitor studies are not considered, the frequency of competitor studies being included, how competitor studies are being graded, the relative frequency of particular drug class reviews, the potential seasonality of drug class reviews, the drugs included and excluded from drug class reviews, the guidelines included, the guidelines excluded, the frequency of various guidelines being included in all DCRs, the guidelines considered, the frequency of investigational agents being cited in drug class studies, the names of investigational agents being cited in drug class studies, real-time alerts when established frequency for a given book is exceeded, blinded conclusion sections, all information organized by geographic regions, all information organized by market channels, correlations of study selections with formulary access, impact of market events (e.g., new indication, new study, new warning) on frequency of drug class, impact of market events (e.g., new indication, new study, new warning) on composition of drug class, study designs ranked the highest by customers (meta analysis, prospective, etc.), study designs which are rarely included (retro), whether the full article or just the abstract is being reviewed or downloaded, what additional related information is being accessed, the number of individuals researching a specific topic, which DCR template or format is being used (if any), timelines (e.g., how soon prior to the scheduled meeting did the evaluation work being), what other information portals were accessed (e.g., NICE, DERP, etc.), and/or other characteristics and insights.

Much of the newly captured data elements are considered as indirect data elements. In other words, in some embodiments, the data has not been selected for storage by the user, but instead indicates the interaction of the user with the system. For instance, a newly captured data element such as “frequency of trials being selected” may be determined based on a number of drugs reviewed for inclusion in the monographs, including those not actually selected. As such, in some embodiments, the actual data selected for input in the generation of a monograph is not used to make a determination of the “frequency of trials being selected.” Instead, the “frequency of trial being selected,” among other data, may be determined by monitored the system's use and ascertaining or extrapolating various statistical information and/or other relevant information. It should be appreciated that the data generated and stored to the activity database 122 may be stored in any suitable format and/or structure.

In block 262, the system 100 (e.g., via a data harvesting application or algorithm) retrieves information from the monograph database 121 and/or the activity database 122 according to one or more predefined rules of extraction. In some embodiments, the system is not only configured to extract desired information from each of the monographs, but is also configured to identify or tabulate the occurrence of certain kinds of information. For instance, a portion of the monographs may be directed to the drug Penicillin. In this instance, the number of monographs directed to the drug Penicillin is tabulated. In other situations, a certain type of information found in a drug monograph may not be necessary, such as whether or not the monograph included the complete product information selectable in the user screen 154 of FIG. 4.

In block 264, the system 100 (e.g., via a data removal application or algorithm) removes confidential information and/or other information/data that would be unacceptable to disclose to third parties. For example, if the harvested data included the name of the individual or entity making the monograph, such confidential information may be removed. It should be appreciated that, in the illustrative embodiment, any confidential, identifying, and/or otherwise sensitive information that is removed does not become part of any final reports. Upon removal of the sensitive information, the data is organized according to categories and selected by the user in block 266.

FIG. 18 illustrates a number of categories that may provide useful information to the user of the final report or to healthcare practitioners interested in patterns of use of healthcare information in making monographs. For instance, a category directed to the drugs included or excluded in the monographs may be identified. A category directed to geographic regions may also achievable from the harvesting of data. The various categories of information provided in FIG. 18, however, is not limiting and is merely representative of the types of information that is obtainable from the data ascertained by monitoring system use (e.g., via the activity database 122) and/or from the database of drug monographs (e.g., via the monograph database 121). Other categories of useful information are contemplated and fewer or greater numbers of categories are possible.

In some embodiments, the user may select data from the organized data for use in a final report. It should be appreciated that one or more templates may be provided and used to provide a customizable report, which may serve to organize the data in a form most useful to a user of the final report. Depending on the particular embodiment, it should be appreciated that the user of the final report may provide the structure of the final report (e.g., a custom or user-specific structure) or one or more templates may be presented for selection by the user of the final report. After the form/structure of the final report is determined, the system may generate the report in block 268. In the illustrative embodiment, the report includes the selected data described above for consideration by the user.

Although the blocks 258-268 are described in a relatively serial manner, it should be appreciated that various blocks of the method may be performed in parallel in some embodiments.

In some embodiments, a customer management screen is available to an administrator or other authorized user and includes one or more tables listing the active customers in the system, as well as customers who have had a subscription and no longer do (“Active”/“Inactive”, respectively), and a pagination control below the table. The illustrative table is comprised of an organization name, a main contact, date of joining, status of inactive or active, an edit organization, and an edit users.

A Disease and Treatment Summary Management screen may be used to manage an internal database that populates a “Disease Summary and Treatment Summary” field. Upon entering this screen, the admin user may be presented with a quick search field and a link to create a new Disease and Treatment Summary pair. Typing a partial string of a disease name may reveal a list of matches (e.g., starting with the second character typed). Selecting a disease from the quick search dropdown list may open the Disease and Treatment Summary pair, pre-filled with the disease's current contents, and above the fields a “Disease Name” title and the last save date. In the illustrative embodiment, these text area fields support tables, since the source database information will contain tabular information mixed with text. A plus sign (“+”) to the right of the disease name may permit the user to add additional disease names to the current disease/treatment pair. In the illustrative embodiment, there is no limit to the number of disease names that can be added. Further, each text area may have a text field directly under it labeled “Disease Summary Citation” and “Treatment Summary Citation”. All fields may be optional except, in some embodiments, there must be at least one (1) Disease Name to save an entry.

In some embodiments, a button group (“Save & Close”, “Save & Remain”, “Cancel & Close”, “Cancel & Remain”) is provided in the editing panel where the user saves or cancels changes. If changes are canceled, in some embodiments, no update is made to the database. If any changes are made in new or existing disease/treatment pair editing panel and the user attempts to select a different disease or create a new one, a confirmation dialog will appear. Clicking “Yes, Continue”, cancels any changes and executes the last command. Clicking “Wait, Let Me Save” closes the confirmation dialog.

In some embodiments, a system process, known as a cron routine, may execute periodically (e.g., each month) to check an [update_date] attribute on all drugs used in all monographs by all users of all roles in the system. If an [update_date] has changed to be a later date than the one currently stored, the system stores the new date in [update_date], records the following information, and compiles the information to populate an email to be sent to a user. For each Set ID updated:

Drug Name

Date Updated

Monograph Affected (count=1)

User Affected (count=1)

User Affected's Email

| Drug Name | Updated On | Set ID | In Monographs | Users Affected |
|---|---|---|---|---|
| [drug_name_1] | [update_date_1] | [set_id] | [#_found_in_1] | [#_users_found_1] |
| [drug_name_2] | [update_date_2] | [set_id] | [#_found_in_2] | [#_users_found_2] |
| [drug_name_N] | [update_date_N] | [set_id] | [#_found_in_N] | [#_users_found_N] |

Two non-expiring GUIDs may be provided for the following reasons: 1) when clicked, the system may send an email to all affected users notifying the users of the drug that changed and the date on which it changed, inviting the users to log-in to the system and re-populate the drug's prescribing info if desired; and 2) when clicked, the stored data may be dumped from the update and GUID #1.

In some embodiments, an email including this information may be sent to all users and the ticketing system containing the compiled list and the two GUIDs, giving the user the opportunity to execute the one preferred. A stateful page may display a message to the viewer depending on the GUID clicked in a Data Update Email. If the first GUID is clicked on the Data Update Email, in the illustrative embodiment, the system sends an email to all affected users with a compiled table of how their account was affected and an invitation to update a user's monograph. In various embodiments, a table includes the drug name(s) and the date(s) updated.

While exemplary embodiments incorporating the principles of the present disclosure have been described herein, the present disclosure is not limited to the described embodiments. Instead, this application is intended to cover any variations, uses, or adaptations of the disclosure using its general principles. Further, this application is intended to cover such departures from the present disclosure as come within known or customary practice in the art to which this disclosure pertains and which fall within the limits of the appended claims.

### APIs for Access to Healthcare Websites and Databases

As described above, the system 100 may utilize various APIs for access to healthcare websites and/or databases. As such, this section provides a description of various embodiments of example APIs that may be included to access the information provided by those databases. As such, in some embodiments, one or more of the APIs described herein may be used in conjunction with the technologies described above. However, it should be appreciated that the APIs are provided by way of example, and the system 100 may utilize additional, alternative, and/or modified APIs relative to those described herein in other embodiments.

### Daily Med API for [Drug Selection]

Using Example Input=“celexa” via https://dailymed.nlm.nih.gov/dailymed/services/v2/spls.xml?drug_name=celexa, this will return three results. Store the Set ID for each. Run a query searching for the <asEquivalentEntity classCode=“EQUIV”> property in each. Exclude the Set IDs are associated with this property. Keep the remaining Set IDs for listing in the drug selection list. In this case there is only one remaining Set ID.

Via https://dailymed.nlm.nih.gov/dailymed/services/v2/spls/4259d9b1-de34-43a4-85a8-41dd214e9177.xmlm, extract the following elements for display in the drug selection list: store <code code=“34391-3”/><title>[ALL Content]</title> as drug_name; if <code code=“34066-1”> exists, store “Yes” as boxed_warning, else store “No”; and store <effectiveTime value=[YYYYMMDD]/> under <code code=“34391-3”/> as update_date.

Via https://dailymed.nlm.nih.gov/dailymed/services/v2/spls/4259d9b1-de34-43a4-85a8-41dd214e9177/ndcs, run a query searching for all instances of the {“ndc”: “ ” } property. Store each as ndc #.

### (Alternative) Daily Med API for [Drug Selection]

The following logic shall be applied to display a list of drugs to user. Once the user inputs full or partial name of the drug, the system shall issue a search query to DailyMed API using the following format: HTTPS://DAILYMED.NLM.NIH.GOV/DAILYMED/SERVICES/V2/SPLS.XML?DRUG NA ME=[INPUT]

The system shall remove all repackages from the list of result by applying the following logic: load detailed SPL for each drug using the following request format: https://dailymed.nlm.nih.gov/dailymed/services/v2/spls/[ID].xml, where ID shall be taken from the initial list of results from <setid> tag; search for <asEquivalentEntity> in the detailed SPL; and if <asEquivalentEntity> tag is found, remove item from the results (as a repackaged drug).

The system shall present a list of the drug names (non-repackaged) to the end user as a drop-down without repeating drug names. The user shall select one of the drug names. The system shall query API using the full drug name. The system shall filter out repackages of the drug. The system shall remove all items except the single most recently published item. Via https://dailymed.nlm.nih.gov/dailvmed/services/v2/spls/4259d9b1-de34-43a4-85a8-41dd214e9177.xml, extract the following elements for display in the drug selection list. Use drug name value from the SPLs list in #2 as drug_name. If <code code=“34066-1”> exists, store “Yes” as boxed_warning, else store “No”. Use SPL latest publication date from the SPLs list in #2 as update_date. Via https://dailymed.nlm.nih.gov/dailymed/services/v2/spls/4259d9b1-de34-43a4-85a8-41dd214e9177/ndcs, run a query searching for all instances of the {“ndc”: “ ” } property. Store each as ndc #.

### Database for [Drug Class Assignment]

In order to display the drug class for an individually selected drug, an equivalency table will need to be created and maintained to permit reverse Set ID association with Drug Classes, not currently available from the DailyMed or OpenFDA APIs. Building the table may require the following information: build a table of drug classes and store <name type> and <code codingSystem> within each <drugclass>, https://dailymed.nlm.nih.gov/dailymed/services/v2/drugclasses.xml, and build drug class code tables from each <code codingSystem> entity and store <setid> within each <spl>. It should be noted that two drugs with unique Set IDs will each belong to a unique Drug Class Name Type, but the drugs may appear to share the same name. Results that appear to be duplicate may not be. It is also possible for one Set ID to be part of more than one Drug Class.

### Database for [DEA Schedule Code]

In order to display the DEA Schedule Code for any drug, an equivalency table will need to be created and maintained to permit reverse Set ID association with DEA Schedule Codes, not currently available from the DailyMed or OpenFDA APIs. Building the table may require the following information: build DEA schedule code tables from the 6 DEA Schedule Codes and store <setid> within each <spl>. https://dailymed.nlm.nih.gov/dailymed/services/v2/spls.xml?dea_schdule_code={code}. The six DEA Schedule Codes are none, c48672 (“CI”), c48675 (“CII”), c48676 (“CIII”), c48677 (“CIV”), and c48679 (“CV”). If SPL contains several DEA codes, minimal DEA should be associated with it. For example, https://dailymed.nlm.nih.gov/dailymed/drugInfo.cfm?setid=fcb16b75-fd8a-4b68-b5e2-8fa9f0b0f88e has CIII and CII. CII should be used.

### Database for LOINC Codes for [Drug Editor]

Currently, LOINC.org is piloting an API, but it is not presently available for commercial use. Therefore, LOINC codes are available via zip file. Within the zip file are several storage methods holding the full list of codes—from csv to mdb, oracle, and sql. The FA system may use and/or identify one or more of the codes within the [SYSTEM] field by “FDA package insert” and “FDA product label”—the only two terms that contain “FDA”. The database can be downloaded and update a subscription to updates can be found at https://loinc.org/.

### Dailymed API for [Drug Editor]

DailyMed APIs are structured to have two main sections: “<author>” and “<component>”. Within the parent “<component>” attribute other “<component>” attributes, several layers deep. Normally this wouldn't be machine readable, but each layer has unique values to determine their order. The level 1<component> has no usable attributes within it, so our search will be on subsequent levels. The level 2<component> contains the main section content. This layer will be used. The level 3 and deeper <component> contains sub section content. These layers will be used. Under each <component> on any level, is a LOINC code attribute (see #2 below). This is the section content.

An example drug Set ID is 4259d9b1-de34-43a4-85a8-41dd214e9177 (celexa). Call the drug from the DailyMed API. https://dailymed.nlm.nih.gov/dailymed/services/v2/spls/4259d9b1-de34-43a4-85a8-41dd214e9177.xml. Find the location of all LOINC codes and store this for reference. LOINC codes are always within <code code=“[loinc_code]” codeSystem=“2.16.840.1.113883.6.1” displayName=“[section_name]”/>. Store the <title> attribute for each LOINC section as [section title]. Within each level 2<component> is html-formatted text in a <highlights> section—which may or may not exist. If it exists, the entire content should be stored as [section_highlights]. Within each level 3 and deeper <component> is html-formatted in <text> sections. This should be stored as [section_content].

### Pubmed API for [Literature Search]

Using an example search=“celexa”, https://eutils.ncbi.nlm.nih.gov/entrez/eutils/esearch.fcgi?db=pubmed&term=celexa&retmax=50 this will return 28 UIDs in the result. Assume the result returned 2 UIDs and take these two UIDs and search for their summary information to populate the table. https://eutils.ncbi.nlm.nih.gov/entrez/eutils/esummary.fcgi?db=pubmed&id=27176755,2677157 2

Each UID's information is wrapped in a <DocSum> attribute. The <Id> attribute within the <DocSum> envelope can confirm which UID is being queried. <Item Name=“PubDate” Type=“Date”>{pub_date}</Item> should be stored as [pub_date]; <Item Name=“PubType” Type=“String”>{pub_type}</Item> should be stored as [pub_type_#]; each item in <Item Name=“Lang” Type=“String”>{language}</Item> attributes should be stored as [pub lang#]; <Item Name=“Title” Type=“String”>{title}</Item> should be stored as [pub title]; and <Item Name=“NlmUniqueID” Type=“String”>{id}</Item> should be stored as [nlmid].

Take these two UIDs and search for their abstract, found within the full content via a json call. https://eutils.ncbi.nlm.nih.gov/entrez/eutils/efetch.fcgi?db=pubmed&id=27176755,26771572. Each UID's information is wrapped within a Pubmed-entry::={“abstract” }, attribute. The abstract attribute will be multiline text and should be stored as [pub_abstract]. Using [nlmid] from #2 above, links to the actual articles are found in the NLM catalog. http://www.ncbi.nlm.nih. gov/nlmcatalog?term=9100907%5BNLM%20Unique %20ID %5D http://www.ncbi.nlm.nih.gov/nlmcatalog?term=0200525%5BNLM %20Unique %20ID %5D

Each link should be stored as [nlm_link]. Using the archived UIDs from step 3, the Elink API is used to search for similar articles. Each UID contains a PMID equivalent wrapped within a Pubmed-entry::={pmid [pmid_#]}, attribute. The pmid # attribute will be a single line of text and should be stored as [pmid]. Using the PMID, search for the “neighbor score”, a similarity score computed by PubMed and available for use via the Elink API. https://eutils.ncbi.nlm.nih.gov/entrez/eutils/elink.fcgi?dbfrom=pubmed&db=pubmed&id=20210 808&cmd=neighbor score. Use the UIDs from that call as the source for the new search results.

### Pubmed API for [Citation]

Using the efetch call from PUBMED API FOR [LITERATURE SEARCH], one can obtain the information necessary to create a bibliographical citation for all rows included in the “Relevant Studies Selected” table. Take these two UIDs and search for their summary information to populate the table. https://eutils.ncbi.nlm.nih.gov/entrez/eutils/efetch.fcgi?db=pubmed&id=27176755,26771572

Each UID's bibliographical information is wrapped in a <DocSum> attribute. Each author is in the cit {authors {names std {name ml attributes and each should be stored as [pub_author_#]. The year of publication is found within the cit {from journal {imp {date std {year attribute and should be stored as [pub_year]. The citation title is already stored from PUBMED API FOR [LITERATURE SEARCH]. The name of the publication can be found within the cit {from journal {title {name attribute and should be stored as [pub_name]. The journal volume can be found within the cit {from journal {imp {volume attribute and should be stored as [pub_vol]. The journal volume's issue number can be found within the cit {from journal {imp {issue attribute and should be stored as [pub_vol_issue]. The pages where the article can be found is found within the cit {from journal {imp {pages attribute and should be stored as [pubpages]. It should be noted that this is only one kind of citation—that of a journal article. Many other types of reference material are available and a set of API calls may be made for each one.

### MMIT API for [MMIT Data]

This API requires a token. Details on generating a token are provided below and further details are found at https://api.mmitnetwork.com/Home/QuickStart#Authentication. When a token is generated and returned, it is accompanied by an expiration. The day before the expiration date, an additional token, in different embodiments, is generated, stored, and used until the day before the next expiration date. The syntax for retrieving a token is:

curl -X POST

-H “Accept: application/j son”

-d {“grant type”: “password”,

“username”:“[FA username]”,“password”:“[FA_password]” }

https://api. mmitnetwork. com/Token An example response of the token request is:

HTTP/1.1 200 OK
Content-Type: application/json;charset=UTF−8
X-FormularyApi-Version: 1.0.0
{
“access_token”:“2XkdxcyewlkdkcxvXelcw232mnD0211x...”
“expires_in”: 199219
“token_type”: “bearer”
}

With a token stored, it can then be used to authenticate a session and, in turn, API calls can be used to get MMIT data via the MMIT API. Start with the NDC numbers we retrieved from DAILY MED API FOR [DRUG SELECTION]. With these we can obtain the MMIT ID and access the desired data.

Much of the information to be retrieved from the MMIT database is static and is to be updated monthly:

the list of “States”: https://api.mmitnetwork.com/Formularv/v1/Geographic/States;
the list of “Channels”: https://api.mmitnetwork.com/Formulary/v1/Plans/Channels;
the list of “Parents”/controllers: https://api.mmitnetwork.com/Formularv/v1/Parents;
the list of “DrugListTiers”: https://api.mmitnetwork.com/Formulary/v1/Coverage/DrugListTiers;
the list of “UnifiedTiers”: https://api.mmitnetwork.com/Formulary/v1/Coverage/UnifiedTiers.

In a single drug review, retrieve and store the “ProductId” and “Name” values for the drug (ex. Celexa). https://api.mmitnetwork.com/Formulary/v1/Products?Name=celexa “ProductId”=78423, “Name”=“Celexa 20 mg tab”. In a multidrug review, retrieve and store the “ProductId” value for all drugs (ex. Ibuprofen, Celexa) where “IsGeneric”=false. Note that ibuprofen is generic and will have more than one “ProductId”, thus the user will have to be presented with a list of drug options from which to choose. This is where “IsGeneric” comes in. Only those “ProductId”s in the list where “ProductId”=false are to be presented to the user for selection/inclusion in the MMIT area. https://api.mmitnetwork.com/Formulary/v1/Products?Name=ibuprofen; “ProductId”=112294, “Name”=“Ibuprofen Cold-Sinus(with PSE)”; “ProductId”=77477, “Name”=“ibuprofen (bulk)”; “ProductId”=142854, “Name”=“Ibuprofen PM (tab)”; “ProductId”=126907, “Name”=“Ibuprofen Cold”; “ProductId”=141867, “Name”=“ibuprofen-diphenhydramine HCl”.

For each “ProductId” found, the user may choose to select/include it into the MMIT section. (Thus, no selection is permissible.) In this case, assume the user selects 78423 (Celexa) and 77477 (ibuprofen (bulk)). The user then selects which state and channel their interested in. Also, assume the user has selected Florida as the state of coverage (FL) and Commercial as the channel (1).

With the “ProductId”, “ChannelId”, and “StateId”, a search can be made via the MMIT API, returning the necessary information to build the first page of the “MMIT Data” report—the “ControllerCoverages” pull. https://api.mmitnetwork.com/Formulary/v1/Coverage?ProductId=77477&ChannelId=1 & StateId =FL. Information from the ControllerCoverages file will be used to build a drug's MMIT report summary: [statement] is at the bottom of each product pull and will be the header for the drug's report; “[#] plans” is an overall number of plans in the current search and requires calculation; <graph/chart> will be a visual representation of the “[#] plans” broken down by “unifiedtiertype”; “unifiedtiertype” will be a list of unified tier types that contribute to the “[#] plans”; <payertable> will be a table of payers with plan and coverage information; and [statement] is found at the bottom of the ControllerCoverage file, as “Statement”.

The “[#] plans” is calculated by using ControllerCoverages and sum all “PlanCount” values. Further, <graph/chart> is calculated by using “ControllerId” (from ControllerCoverages), “ChannelId”, and “StateId”—the “Plans” pull. https://api.mmitnetwork.com/Formulary/v1/Plans?ControllerId=3164&ChannelId=1 & StateId=F L. Store all “PlanId” values. Use each “PlanId” from the Plans pull with “ProductId”—the “PlanCoverages” pull. https://api.mmitnetwork.com/Formulary/v1/Coverage/Plan?ProductId=77477&PlanId=5429 Store “UnifiedTierId” value for each PlanCoverages pull. Match each “UnifiedTierId” value with its “Color” values in the UnifiedTiers file. Draw the “unifiedtiertypes” graph. Each unique UnifiedTierId should have its own bar in the chart, colored with its “Color” value and be drawn in descending order from left to right. Each bar in the chart should represent the number of times that unique “UnifiedTierId” value occurred for all “PlanId”s for all “ControllerId”s.

The “unifiedtiertype” is determined by drawing the “unifiedtiertypes” graph and legend. Match each “UnifiedTierId” value with its “Name” values in the UnifiedTiers file. Show each unique UnifiedTierId Name in the legend, colored with its “Color” value obtained from <graph/chart>.

The <payertable> is determined in a manner that the <payertable> table will have three columns. Sort by Payer/PBM: Payer/PBM; # of Plans; Status.

The “Payer/PBM” is determined by using “ControllerId” from ControllerCoverages, match all “ControllerId”s with their “Name” value from the Parents file, and display the “Name” value for each, one per table row.

The “# of Plans” is determined by using ControllerCoverages, display the “PlanCount” values for each table row.

The “Status” is determined by using the Plans pull (store all “PlanId” values and store all “Lives” values), using PlanCoverages pull for each PlanId (store “DrugListTierId” value and store “UnifiedTierId” value), and determining coverage status (sum all “Lives” for each row (“ControllerId”) and divide it by the number of covered “lives”; and display as a percent with “Covered” added at the end. Example: “70% Covered”). It should be noted that if “UnifiedTierId” is 1, 2, 3, 4, 5, or 9, the “ProductId” is covered. Further, if “UnifiedTierId” is 6, 7, or 8, the “ProductId” is NOT covered.

It is possible that the user will want to drill down and view coverage information on the contributing plans for the rows of the <payertable>. This extra detail will be referred to as <contributordetail>. The <contributordetail> is determined in a manner that the <contributordetail> table will have three columns: Plan, Status, and Raw Status. Sort by Status, then Plan. Status sort by “UnifiedTierId”: 3, 4, 5, 1, 2, 9, 6, 7, 8.

The “Plan” is determined in by using Plans, display “Name” for each “PlanId”, one per table row.

The “Status” is determined by using PlanCoverages, match the “UnifiedTierId” with its “Name” in the UnifiedTiers file.

The “Raw Status” is determined by using PlanCoverages, match the “DrugListTierId” with its “Name” in the DrugListTiers file.

### OPENFDA API for [Clinical Trials]

The API for ClinicalTrials.gov is REST-based and returns a full webpage worth of data, as per usual. A full URL that includes all variables possible is below and will return a result of every study in the database. https://clinicaltrials.gov/ct2/results?term=&type=&rslt=&recr=&age_v=&gndr=&cond=&intr=& titles=&outc=&spons=&lead=&id=&state1=&cntry1=&state2=&cntry2=&state3=&cntry3=&lo cn=&rcv_s=&rcv_e=&lup_s=&lup_e=

All variables, in various embodiments, are included with each call to the API, even if blank. However, there are accepted arguments for each variable. All fields, checkboxes, and dropdowns are optional. If more than one checkbox is selected, the variable is repeated with the additional argument. They include term=(text field), type=(dropdown), rslt=(dropdown), Recr=(dropdown), age_v=(text box), age=(3 checkboxes, can be used with [age_v]), gndr=(dropdown), Hlth=(checkbox), cond=(text field), intr=(text field), titles=(text field), outc=(text field), spons=(text field), spons_ex=(checkbox), lead=(text field), lead_ex=(checkbox), id=(text field), phase=(5 checkboxes), fund=(4 checkboxes), rcv_s=(formatted text box [mm/dd/yyyy], rcv_e=(formatted text box [mm/dd/yyyy], lup_s=(formatted text box [mm/dd/yyyy], and lup_e=(formatted text box [mm/dd/yyyy].

Further, the term=(text field) can be any word or words, does not require quotes, words are separated by a “+” character, boolean operators in CAPS are permitted, multi-word operators are permitted with parentheses, and parentheses are converted into their ASCII equivalents-%28 and &29. The type=(dropdown) can include [null] (returns all results as throughout this section), Intr, Obsr-> PReg, and Expn. The rslt=(dropdown) can include [all], With, Without. The Recr=(dropdown) can include Open (Not+yet+recruiting, Recruiting, Available) and Closed (Active %2C+not+recruiting, Completed, Terminated, Suspended, Withdrawn, Enrolling+by+invitation, Temporarily+not+available, No+longer+available, Approved+for+marketing, Unknown+status). The age_v=(text box) can accept any positive integer. The age=(3 checkboxes, can be used with [age_v]) can include 0 (returns results for “child” range), 1 (returns results for “adult” range), and 2 (returns results for “senior” range). The gndr=(dropdown) can include [all], Male, and Female. The Hlth=(checkbox) can include Y (selected). The spons_ex=(checkbox) can include Y (selected for exact match of [spons]). The lead_ex=(checkbox) can include Y (selected for exact match of [lead]). The phase=(5 checkboxes) can include 4 (phase 0), 0 (phase 1), 1 (phase 2), 2 (phase 3), and 3 (phase 4). The fund=(4 checkboxes) can include 0 (NIH), 1 (US), 2 (Industry), and 3 (Other).

### Monograph Editor

At least one embodiment of a monograph editor described and/or referenced above is described in greater detail below. As such, in some embodiments, one or more of the features of the monograph editor described below may be used in conjunction with the technologies described above. However, it should be appreciated that the monograph editor details described below are provided by way of example, and the monograph editor of the system 100 may include additional, alternative, and/or modified features or functions relative to those described below in other embodiments.

### Monograph Editor: “Create”

A “Drug Classification Reference” field is a dropdown list connected to an internally managed data table which permits a single selection. There are currently 3 drug classes; “USP-DI”, “AHFS”, and “CMS”. The user may select any option from this list, including a fourth labeled “Custom Drug Class”. Upon selecting “Custom Drug Class”, a light box dialog will be displayed for the user to name their own drug class. The user can back out of this process by clicking the “Cancel” link or create the custom drug class reference by clicking the “Okay” button.

Once created, the custom drug class reference name will be displayed in the dropdown list, permanently for that organization. A “Create Custom Class” option will still remain in the dropdown, permitting the organization users to create as many classes as desired, visible to all within the same organization. When a custom class reference is displayed as the selected item, an “Edit” link will appear to the left of the dropdown list. Clicking the “Edit” link will bring up the custom class light box dialog with the custom class reference name appearing in the text field. The user can edit the name as desired and either click “Cancel” to back out of the renaming process or click “Okay” to save their changes.

A single field begins the actual drug monographing process. The user types in a drug name in order to begin seeing the drugs available for selection. In order to ensure that the correct drug list and latest information is being referenced, the system calls an API and the required tertiary data sources.

Each listed drug contains four data elements and an “Add” icon. The data elements, in order, and where they are found include: drug name, drug type, NCD codes, boxed warning (yes/no), and last updated.

Once the correct drug has been found, the user clicks an “Add” icon in order to add it to the monograph. In one embodiment, clicking the “Add” icon for the first selected drug in the monograph will present the user with two options: “Select for Single Drug Review (you can add more later)” and “Select and Add Another Drug”. Clicking the first option will immediately take the user to tab 2 of the monograph editor, “Populate”. In another embodiment, the must decide on a single drug or a class of drugs at the outset and, therefore, the user is either taken into the monograph editor for a single drug or a class of drugs based on the user's selection.

Clicking “Select and Add Another Drug” will keep the drug selection window visible and the current search criteria will remain in the “Drug” text field. When the user has found a subsequent drug to add to the monograph, a light box dialog will be displayed for the user, stating “It looks like you're selecting more than one drug. Do you want to filter by class for a class review?” and prompting the user to select one of three responses.

If the user selects “No”, the drug will be added to the monograph and the user will remain at the drug selection window and the current search criteria will remain in the “Drug” text field. At each drug selection with a “No” response, this will be the workflow.

If the user selects, “No. Don't ask again for this monograph,” the drug will be added to the monograph and the user will remain at the drug selection window and the current search criteria will remain in the “Drug” text field—just like the “No” response. However, at each drug selection, the drug will be added to the monograph and no light box dialog will reappear. This is a user-level selection and does not apply to all users who have access to the organization's monographs.

However, if the user selects “Yes”, a drug class selection area will be displayed and the drug selection area will be minimized under it.

The Drug Class Selection area presents the user with a quick search field much like the drug search field, except this search field filters the list of displayed drug classes that are below it. Additionally, a set of 5 checkboxes allows the user to filter the list of drug classes by their class categories.

“All” is selected by default and displays all drug classes in the list box, where the user can add drug classes using the “Add” icon to the “Selected Drug Classes” list on the right. Each selected drug class in the “Selected Drug Classes” will have a “Remove” icon to remove it from this list and return it to the list of available classes.

The other four checkboxes filter the list of drug classes in the same way as the “All” checkbox. However, when one or more of them is selected all applicable classes are displayed in the list box and the “All” checkbox is cleared. When the “All” checkbox is selected, each of these four checkboxes is cleared. If all four of these checkboxes are selected, they will be cleared and the “All” checkbox will be selected. It is possible that not all users will know what these drug class category abbreviations mean. Hover text may be used to display the full drug class category name of each.

Once the user feels they have a complete list of “Selected Drug Classes,” the user can click the “Use These Classes” button, which will minimize the drug class selection area and display the drug selection area below it. The user can toggle between these areas by clicking on the drug class areas minimized bar and the “Use These Classes” button.

If any monograph has ever been approved by a user, the system may apply the included sections to any new monograph started, by type, for that user. That is, if a new single-drug monograph is started, the “Included” checkboxes should be set to selected and unselected based on what the last-approved single-drug monograph was set to, and the same applies with a class monograph.

Each time a new monograph is created and included sections are applied based on a prior approved “same type” monograph, a note may appear at the top of the Create tab notifying the user and may state, “Settings of included and excluded sections have been applied from the most recently saved monograph. Please adjust if necessary.” An “X” may be visible at the right side of this note so the user can close it for that monograph.

### Monograph Editor: “Populate”

Each drug may be selected from a tab of the monograph editor and appears as a list of minimized bars, unless the user has selected only one drug in which case the drug's “Populate” editor is maximized with no opportunity to minimize the contents.

When more than one drug is listed on this screen, each minimized bar contains the drug name, classification reference, an editing note to the user, a status of the drug with the date of its last edit, and “up” and “down” icons so the user can re-order the list as desired. The status of “Completed” or “In Progress” is determined by counting the selection of “Completed” and “Include” checkboxes. If the ratio of “Completed” to “Include” checkboxes is 1:1, the status is “Completed”. If the ratio is different, the status is “In Progress”. If the number of “Include” checkboxes is 0, the status is “Not Started”. For “Completed” and “In Progress” drugs, the date of its last edit is displayed in a small font below its status, right justified.

Clicking the minimized bar expands the drug editor. In some embodiments, only one drug editor at a time can be maximized. The formerly minimized bar changes slightly in that the editing note to the user is hidden. The “up arrow” and “down arrow” icons become unavailable for all drugs. What remains is the drug name, classification reference, and drug status—the drug editor title bar.

Under the drug editor title bar is an area for summary information. Each listed drug contains four data elements and an “Add” icon. The data elements, in order, and where they are found include: drug name, drug types, NDC codes, boxed warning (yes/no), DEA Schedule; last updated; and Drug Class.

In a drug summary area, a set of buttons are included to save or cancel editing a drug in different ways as follows: “Save & Minimize” will save all edits and minimize the drug editor; “Save & Refresh” will save all edits and remain on the current drug editor; “Cancel & Minimize” will ignore edits and minimize the drug editor; “Cancel & Remain” will ignore edits and remain on the current drug editor. Minimizing a drug editor includes the “up arrow” and “down arrow” icons, permitting the user to re-order the drugs. This will be reflected in this list and the final outputted document.

All content in each drug editor may be obtained from XML via an API. The mappings as used in the editor include the field “Text from ‘Use Highlights (FA Recommendation)’” with the field type of “Read-Only Text Area”; the field “Text editor from ‘Use Highlights (FA Recommendation)’” with the field type of “Editable Text Area”; the field “Text from ‘Use Full Prescribing Instructions’” with the field type of “Read-Only Text Area” and the field “Text editor from ‘Use Full Prescribing Instructions’” with the field type of “Editable Text Area.”

Above and below the group of sections is a text link that reads “Expand/Collapse All Populated Sections”. Clicking this will expand only those sections which have been selected from either the unedited highlights, the unedited full prescribing instructions, or custom (edited) text content. Section titles may differ based on the age of the source data. It has been found that some drugs contain section numbers and some do not. Sections are to be imported into this screen in the order in which they are provided in the source data

If the drug has a Boxed Warning, it will be displayed the first time the drug editor is maximized. After that, the last view of this section will be the default view the subsequent view of the editor. The Boxed Warning section is always completed. In fact, the “Completed” checkbox is not even provided. However, the Boxed Warning section can be included or excluded from the monograph by use of the “Include” checkbox. However, the content of the warning itself cannot be edited. The Boxed Warning title contains a reference asterisk. At the bottom of the maximized drug editor is the reference text that reads, “*Even if not including the Boxed Warning section, a note will appear on the final Monograph stating that a boxed warning exists for this label.”

The content in all other sections of the drug editor can be edited. Each section, besides the Boxed Warning, can be fully manipulated by the user, except for the section title. Below the section title are a pair of radio buttons that tells the section what to display. The “Use Highlights (FA Recommendation)” radio button will expand the area below the radio button to reveal that section's highlights as read-only, formatted text.

The “Use Full Prescribing Instructions” radio button will expand the area below the radio button to reveal that section's full prescribing instructions as read-only, formatted text, minus the highlights. The user can switch back and forth between these two radio buttons and doing so will uncheck the “Completed” checkbox, requiring the user to approve the change.

In one or more embodiments, a section may not have a highlights attribute. If this is the case, the radio button for this option is disabled and the text label may be grayed out. Each section can also be expanded or collapsed by use of an expand/collapse icon to the left of the section title. If a section expanded for the first time using this icon instead of one of the radio button, the “highlights” may be selected displayed by default if they exist. Otherwise, the “full PI” may be selected and displayed.

Once the user has selected either the “highlights” or “full PI” radio button, an “Edit” link is displayed to the left of them. Clicking the “Edit” link hides itself permanently, unselects the “Completed” checkbox for the section, converts the read-only text into editable text within a WYSIWYG-capable editor, and un-hides the “Custom Text” radio button and selects it. The “Include” checkbox selection status is unchanged. This three-button configuration allows the user to switch between the highlights, full PI, and editable version of the section content.

A view state (expanded/collapsed) of each section persists for the user (e.g., not organization) through the drug's minimization/maximization, changing of screens, and even logging out/in.

### Monograph Editor: “Support”

Each drug may be selected from a tab of the monograph editor and appear as a list of minimized bars, unless the user has selected only one drug; then the drug's “Summary” editor is maximized with no opportunity to minimize the contents.

When more than one drug or class is listed on the screen, each minimized bar contains the drug name, classification reference, an editing note to the user, a status of the drug with the date of its last edit, and “up” and “down” icons so the user can re-order the list as desired. The status of “Completed” or “In Progress” is determined by counting the selection of “Completed” and “Include” checkboxes. If the ratio of “Completed” to “Include” checkboxes is 1:1, the status is “Completed”. If the ratio is different, the status is “In Progress”. If the number of “Include” checkboxes is 0, the status is “Not Started”. For “Completed” and “In Progress” drugs, the date of its last edit is displayed in a small font below its status, right justified.

Clicking the minimized bar expands the text editing boxes. In some embodiments, only one text editor at a time can be maximized. The formerly minimized bar changes slightly in that the editing note to the user is hidden. The “up arrow” and “down arrow” icons become unavailable for all drugs. What remains is the drug name, classification reference, and status—the text editor title bar.

When maximized, two text areas are presented, “Disease State Summary” and “Drug Class Summary”, each with its own “Include” and “Complete” checkboxes. If data for the selected drug exists in the internal DSS/DCS database, that information may be pre-populated here for further editing. If data for the deleted drug(s) do not exist, a GUID may be generated by the system and inserted into an email to be sent to the system administrator.

### Monograph Editor: “Evaluate”

The “Evaluate” tab may replicate the feel of the PubMed search results screen (see www.ncbi.nlm.nih.gov/pubmed/?term=celexa, for example). The Literature Search section is pre-populated for its first use by doing a simple, search against the PubMed API. Each word in this “pre search” comes from the list of drug names from a tab of the monograph editor. Each time a search is performed, including the pre-search terms, it is displayed as a row in the “Recent Searches” light box flyout that slides into view from the underside of the simple search field. In one embodiment, search results are limited to 500 records and displayed as a list with the attributes and properties such as: system-generated reference number, add/remove/added and hide/unhide buttons, literature title, list of authors, citation, and a link to perform a search for similar articles.

The search results are displayed in the order they are received from the PubMed API. If the user wants to hide a result from view, the user clicks clicking the “Hide” icon. In one embodiment, this table always hides manually hidden studies and results older than 5 years and paginates at 10 rows, not typically less (unless the filtered search result itself is less).

In various embodiments, searches are filtered by the standard list of filters available in PubMed. Searches appear in a stylized “Filter for” box, listing all of them, which sits in a fixed location between the search flyout tabs and the search results. The default list of filters includes those shown on the prototype, as well as those not selected to be displayed in via a pop-up that appears when clicking a filter section's “Customize” link. When displayed, any additional filters selected will be added to their respective list when the user clicks the “Show” button. The user may also cancel the action of adding filter options by clicking the “X” in the upper right-hand corner. The selection of filters acts as a compound filter (using AND) and not an inclusion filter (using OR).

The PubMed search utility itself is displayed by default as a search area to the right of the top page buttons. The simple, single-line, multi-field search tool is shown. Performing a simple search is easy—insert the term(s) and click “Search”. Once clicked, the flyout will re-hide and the page will display the search results.

When searching, the search results appear prominently at the top of the list of results in the Evaluate tab. Additionally, a paginator may be added to the top of the list that appears on the Evaluate tab.

If the simple search is insufficient for the user, the “Advanced Search” text link can be clicked. Doing so will reveal a replica of the PubMed advanced search area, including the ability to select the field(s) in which to search and the ability to add any number of these detailed query options. From the advanced search view, the user can go back to the simple search view by clicking the “Simple Search” text link or perform the search by clicking the “Search” button. Like the simple search, once the “Search” button is clicked, the flyout will re-hide and the page will display the search results.

The publication title may be linked to the actual publication abstract, obtained from [pub_abstract]. When the user clicks linked title text, a light box dialog may appear, displaying the abstract in a formatted window. The top of the window contains an action bar with a “Show Additional Info” button, a “select” or “remove” icon (depending on the table the user used to call the abstract), a “hide” or “unhide” icon (again, depending on the state), and a “Close” button. This action bar is separated from the abstract text by a horizontal line.

In the abstract light box window, clicking the “Show Additional Info” button will open a new window to [nlm_link] not a new tab. If the user opened the abstract from the search results table, the “select” icon will be displayed. If the user opened the abstract from the table of selected studies, the “remove” icon will be displayed. If the abstract belongs to a study record that's not hidden, the “hide” icon will be displayed. If the abstract belongs to a study record that's hidden, the “unhide” icon will be displayed. Clicking “Close” will close the abstract.

Clicking the “select” icon either in the list of search results or the abstract light box, will add that search result to the “Relevant Studies Selected” table that sits below the bottom buttons. The selected study will remain in this table unless it is removed by clicking the “remove” icon either in the table or the abstract light box, which can be viewed by clicking the publication title.

The “up arrow” and “down arrow” icons in the “Relevant Studies Selected” table permit the user to re-order the publications to their preference. This will be reflected in this table, the same “Relevant Studies Selected” table, and the final outputted document.

### Monograph Editor: Compare

For new monographs, a compare screen may be comprised of five dynamically editable tables: a Literature Comparison Table; Summary of Incremental Cost-Effectiveness Ratios; Validation of Instruments; Cost-Effectiveness Evidence Summary; and Methodology of This Review.

In one or more embodiments, the screen can be very “long” so each table is in a container, much like each drug or the DSS/DCS data. If the user chooses to do nothing on this screen, the “Save & {step}” buttons will still work as normal—the next time this screen is loaded, it will be as if they entered this screen for the first time, and so on if they continue with no editing. A “Skip for Now” button will simply open the next monograph tab.

Every table is fully editable and operates the same way. In various embodiments, the features may include: columns can be added by clicking the green “+” at the top-right end of the table; columns can be deleted by clicking the red “x” to the left of the column name; rows can be added by clicking the green “+” at the bottom-left end of the table; rows can be deleted by clicking the red “x” to the left of the row name; deleting rows or columns requires yes/no confirmation from the user: “Are you sure you want to delete this [“row”/“column”]? The data will be lost.”; headers can be edited by clicking on the header name; cell content can be edited by clicking on the cell; a set of save/cancel buttons, like other tabs, is provided for each table; “Save & Minimize” will save all edits and minimize the drug editor; “Save & Refresh” will save all edits and remain on the current drug editor; “Cancel & Minimize” will ignore edits and minimize the drug editor; “Cancel & Remain” will ignore edits and remain on the current drug editor; an “Include” checkbox is provided to display or not display the table on the final report; a “Complete” checkbox is provided to confirm that the table is complete; up and down arrows are provided to allow the user to rearrange the order of tables; tables are minimized on screen load, the words “Click here to show the table” are provided under the table title, a table is maximized/shown by clicking the front part of the minimized bar, not just the word “here”, but a large area—the entire area from the far left of the bar to 20px to the left of the “Complete” checkbox; the height of each row will expand with cell that has the most text; the width of each row may equal the page width (web page or PDF page) divided by the number of columns; all cells created by adding a row or column may contain “Edit/Delete Text . . . ”; all headers with no pre-provided text may contain “Edit Name”; all cells deleted by deleting a row or column will have their data permanently deleted, only retrievable by the user clicking “Cancel & Minimize” or “Cancel & Remain” for the table, “Cancel & Leave” for the page if the table has not been saved, or “Skip for Now” if the table has not been saved.

The Literature Comparison table includes an additional function. The user is presented with two radio button options to the right of the table's title: “Standard Headers (Formulary Academy Recommendation)” and “Custom Headers”. Selecting the “Standard Headers { . . . }” button will present a table with pre-filled headers. The headers of this table are Citation, Evidence Grade, Design/Duration, Population, Interventions, Primary and Secondary Endpoints, Outcomes, and Conclusion/Limitations.

The Citation column will be automatically populated with citation information from an API containing the following text string and, like all other cells, is editable. An empty row for each study in the “Relevant Studies Selected” table may be drawn when the user first selects the “FA Recommended” table. An “abstract” icon for each auto-created row is provided between the citation column and the row's red “x”. When clicking this icon, the abstract appears in a moveable child window, permitting the user to easily copy/paste its contents from the abstract into the table without having to open and close the abstract.

If the user deletes a row that has an abstract associated with it, the normal confirmation dialog listed in the standard table functions will be presented. However, if the user wants to add a row to the table after having deleted one or more rows with an associated abstract, a light box dialog will be presented to the user, asking them to select a previously deleted row with its associated abstract from a dropdown list, or to create a new blank row.

If the user wants to add a row and has not deleted any rows with associated abstracts, no dialog is presented and a new blank row is added to the table.

If the user selects the “Custom Headers” radio button, a table with one column and one row will be presented. If the user changes from “FA Recommended” to “Custom Headers” or vice versa, the data in the hidden table is preserved, allowing the user to change back to it via the radio buttons. Finally, an “Evidence Grades” legend is provided below the table for reference.

An “Evidence Grades” column, visible if “Standard Headers” is selected, will contain a dropdown list of available evidence grades for selection. Below the evidence grades will be “CUSTOM TEXT” and “None or N/A”. If the user selects “CUSTOM TEXT”, the cell will change into a free text field for user input. Next to the free text field will be an icon which will allow the user to change back to the dropdown list if they choose. Clicking the icon will display a warning dialog letting the user know that continuing will change the cell back to a dropdown list and the custom text, if any, will be lost. The user may then confirm or cancel the warning. If confirmed, the dropdown list will re-appear and the selected item will be the top of the list, “Select . . . ”

A Summary of Incremental Cost-Effectiveness Ratios table is present and includes unique features and a special function. First, a “Cost/QALY (USD)” label sits at the top of the table for reference. Next, the header row is named “REFERENCE”, which does not change. Under this, labels for groups of rows are displayed. Labels are editable by clicking on the text, just like any other cell, but their style is that of a header.

A row group permits more than one row to be visually associated with other rows next to it and of its type. Row groups are separated by a horizontal line to ensure clear delineation. Row groups are added by clicking the green “Add Row Group” “+” icon. When clicked, a new row is added, along with a row group title. The user may add rows to a row group by clicking the “Add Row” “+” icon placed within the group. Adding a column adds a column down the entire table, without consideration of row groups.

Row groups can be deleted by clicking the red “x” to the left of the row group title. When a row group is deleted, all rows within the group are also deleted. The same deletion confirmation dialog may be presented to the user as for deleting a single row.

A Validation of Instruments table has pre-provided column header names. However, the names are still editable and deletable. At the bottom is a reference to a column name: “M.I.D.”. Although a user can edit the MID column name, the reference may remain.

A Cost-Effectiveness Evidence Summary table has pre-provided column header names and are editable and deletable. At the bottom is an abbreviation reference.

A Methodology of This Review table has pre-provided column header names and are editable and deletable.

One or more custom tables are made using a button below the tables, but above the save/cancel buttons, labeled “Add an Additional Table”. The user clicks and add a custom table to the bottom of the list. Any number of tables can be added. All normal table functions apply to a custom table, except the user can provide a table title in the “Table Title” field.

If the user chooses to delete the table, they can do so by clicking the “Delete Table” button to the right of the “Table Title” field. Clicking this button will display a confirmation dialog, requiring a yes/no response from the user. Data in a deleted table is handled just as data in a deleted row or column, as described in the standard table functions at the top of this section.

When adding a study to the table, it's not clear that the addition has been made—especially on smaller screens where there are many results. Each time a study is added, a notification bar may appear to the right of the “Cancel/Save/Save & Next” buttons making it clear that the study has been added to the Selected Studies table at the bottom of the page. The bar may appear for five seconds, then disappear. The user can click an “X” to the right of the bar to close it earlier.

If studies are added in rapid succession, the notification bar may be hidden before subsequent ones are shown. In the prototype, you can see this done by clicking the three studies in rapid succession. When the second and third studies are selected, they hide the notification before showing it again.

### Monograph Editor: “Summaries”

A “Summaries” tab is made up of text areas. First, an Executive Summary section is a WYSIWYG-capable text editor which loads default content with the page and selects the “Use the Formulary Academy Executive Summary Template” radio button. If the user wishes to create the executive summary on their own, selecting the “Create an Ad Hoc Executive Summary” will clear the text area.

If the user chooses to click the “FA Template” radio button after having edited the content from creating an ad hoc executive summary, the system will display a browser dialog that reads “If you change back to the Formulary Academy template, you'll lose what you've done on your own. Are you sure you want to do this? (You can cancel this action now and copy the content if you want to save it.)” with a “Yes”/“No” response option.

If the user selects “No”, nothing changes. If the user selects “Yes”, the text area contents are replaced with the FA template. Any changes back and forth to the summary type or edits to the text will unselect the “Completed” checkbox, requiring the user to consciously confirm that the changes made are Completed.

A “Disease Summary” and “Treatment Summary” fields will be placed on the Summary tab under the “Summary of Published Literature”. At the top of these two fields will be a quick search field that permits the user to select a disease from the internal database managed. Selection of a disease name will populate text in the Disease Summary and Treatment Summary text areas below, populate each Summary's Citation, and hide the Disease Name quick search field, revealing a button to the right labeled “Add New Disease”. Clicking the “Add New Disease” button will unhide the “Disease Name” field and hide the “Add New Disease” button.

If the user wishes to request an update to an existing disease/treatment summary pair or request a new pair, they may do so by clicking a button to the top-right of the disease name field, labeled “Request Update or New Disease”. Clicking that button will open the user's default email application via the mailto action, including a default To, Subject, and Body using the following syntax (including the period (“.”) at the end):
mailto: cases(@,formularvacademy.fogbugz. com?subject=Disease %20and %20Treatment % 20 Summary%20Request&body=Enter %20your%20request %20here.

The text area fields support tables, since the source database information will contain tabular information mixed with text. All components of this area—the Disease Name, Disease Summary(ies), Treatment Summary(ies), and the Citation fields—may be bounded above and below by a blue bar to indicate its separateness. The user may freely select to include each field, none, any, or all by use of the “Include” and “Complete” checkboxes above each text area. The content of the Disease Name field itself will not be carried into the monograph review screen since the disease name will be part of the Disease and Treatment Summary content. The bibliographical Citations that are included may be added to the Bibliography section of the Monograph Preview and numbered appropriately, making sure it carries over to the exported PDF (and MS Word doc).

The Conclusion section is also a WYSIWYG-capable text editor, but has no default text—it's just empty. The “Completed” field is just a simple date picker, but does have some power. When the “Completed” date occurs, the monograph status automatically moves from “In Progress” to “Completed”.

The “Monograph Title” field is required for a monograph's state to become completed. However, it is not a required field to save a monograph. It's free text, limited only in that the text entered is on a single line.

The next field, “Reason for Review”, is also a free text field. It may contain hint text. It's also limited only in that the text entered is on a single line.

At the bottom of the Summaries tab, below “Monograph Name”, is a dropdown list labeled “Market Channel” with the following options: Institution, Commercial, Medicare, Medicaid, Managed Medicaid, DoD, VA, Other. If “Other” is selected, a text field may be made visible for the user to type up to 255 characters of plain text.

Finally, there's also the ability to add one or more custom text fields if the user wants to by clicking the “Add Custom Text Area” button. Doing so will reveal an area with a blank title field, WYSIWYG-capable text editor, and the standard Include/Approve buttons, as well as a button to remove the section altogether. Custom text areas are placed above the date pickers in the order in which they were created.

### Monograph Editor: “Optional Considerations”

In another tab of the monograph editor, the list of drugs is displayed similar to a “Populate” tab, except that only the drug name and instructions to click the minimized bar are provided. For each drug, three WYSIWYG-capable text areas are provided. Each of them has an associated API that populates data of potential interest.

An “MMIT Data” field obtains its content from [mmit data]. This is rather simple interface for a highly complex API pull/process. A list of drugs (or a single drug) will be shown, giving the user an opportunity to select any, all, or none of them for which to include MMIT data. Once the user has made their selection, they will click a “Generate MMIT Data Reports” button.

Although this information is immediately available in at least one embodiment, a message can be displayed to the user stating, “Thank you! The summary report of MMIT data will be sent to you by this time tomorrow,” and an email will be scheduled for 23 hours from the current time to go to the current user, containing a GUID link directly to the “Optional Considerations” tab.

A GUID link to an external MMIT Data page can also be generated for the user to email, which when clicked will display only this MMIT Data area on its own page. This GUID link can be obtained by clicking a button next to the “MMIT Data” header that reads, “Create Secure External Link to MMIT Data Report(s)”, but this button is only visible once the “23-hour email” is sent. When clicked, a small pop-up will be displayed, prompting the user to copy the GUID link to their clip board, or canceling. Both actions will close the pop-up after a confirmation message.

For each drug selected, the following graph/table combo will be provided: At the top of the MMIT Data section may be the “Statement” retrieved from the API. Below that may be an area displaying a vertical bar graph. Under the bar graph, to the left, may read the number of plans counted, and to the right a legend for the graph. Below the graph area will be the <payertable>. When any row of the <payertable> is clicked, a pop-up child window will display the <coveragedetail> table. This table can be closed by clicking anywhere outside of the pop-up.

The user can modify their selection of drugs in the MMIT Data selection. Doing so will update the MMIT Data area immediately and NOT send the “23-hour email” again. If the user unselects all drugs or elects not to include the MMIT Data section by unselecting the “Include” checkbox, the GUID sharing button will be hidden from use, preventing GUIDs to be sent for blank pages.

Since there is no connection between DailyMed and MMIT, the user's original search terms from Tab 1 are stored and used here to submit to MMIT. For instance, if the user types “celex”, sees what they want from the filtering list, and selects it, at that point, we need to store “celex” and use it in the API call to obtain the “ProductId”, for instance “/Formulary/v1/Products?Name=celex”, which works the similarly to: “/Formulary/v1/Products?Name=celexa” because partial terms are acceptable.

Same thing with branded generics, such as “ibuprofen”. This may return 1121 results from DailyMed, only some of which will be displayed on our list because of the missing “EQUIV” property. Of those remaining, the user will select the drug they want and we need to store their original search term, which would probably be “ibupr” and search for /Formulary/v1/Products?Name=ibupr in MMIT.

The user will then have to re-select the drug they want in the MMIT section. A note may be included that “The exact drug name chosen on the Create tab may not be the same in the MMIT database. Please select the applicable drug from the following list(s), if they're present. If the desired drug does not appear, please modify your original search term here:.”

After this statement, one text field for each original search term may be displayed, containing the original search terms. Below these text fields may be a “Resubmit to MMIT”. When clicked, any selections for UNCHANGED terms may remain unselected. Any selections for CHANGED terms will be replaced anyway. Rules for the “23-hour email” still apply.

In one embodiment, no actual API will be utilized for the FAERS section of the monograph. Instead, a button labeled “Search FAERS” will appear below the text area as shown in the prototype. Also, a note to the user may be displayed below the text area, which reads: “Clicking this button will open the FDA's AERS reporting website in a new window, leaving this window open so that no changes will be lost or saved. The text area above is provided for you to include your findings on FAERS, if you choose.”

The “Clinical Trials” field obtains its content from [clinical trials]. There is no guarantee that this field will always have information available. If nothing is found from the API data pull, a default message may be placed as text in the text area that reads “No data was found for this section.”

An expandable search area will be available for the user to search ClinicalTrials.gov and automatically populate the Clinical Trials field. The minimized search area will contain a text link that reads “Click here for options to search and auto-populate information from ClinicalTrials.gov.” When clicked, the search area will expand, revealing the same layout as the search area at https://clinicaltrials.gov/ct2/search/advanced—specifically the area below “Advanced Search” and above the “Search” button, except the “Locations” area.

At the bottom of the search area will be a “Search” button and “Help” link, just like the one on ClinicalTrials.gov's advanced search page. When the “Help” link is clicked, a pop-up dialog will be displayed, notifying the user that they will be viewing the actual help page at ClinicalTrials.gov and it will open in a new window.

When the “Search” button is clicked, the search criteria will be run via API and the results of that API will be stored, modified, and displayed, in a results area which will expand above a now-minimized search area. A checkbox will be displayed to the left of each result for the user to select one or more to add into the text area via an “Add Selected Studies” button. When added, the information inserted will include the “Rank” and “Study” name, as well as the list of “Conditions” and “Interventions”, followed by a link to the study named: all information returned in the search results.

In another embodiment, an ability to add one or more custom text fields if the user wants to by clicking the “Add Custom Text Area” button. Doing so will reveal an area with a blank title field, WYSIWYG-capable text editor, and the standard Include/Approve buttons, as well as a button to remove the section altogether. Custom text areas are placed above the bottom button bar in the order in which they were created.

1. A method for generating healthcare information based on a plurality of drug monographs, the method comprising:
retrieving healthcare information from one or more databases;
generating a plurality of drug monographs using the healthcare information retrieved from the one or more databases and based on user input from a plurality of users;
monitoring activity of the plurality of users during the generation of the plurality of drug monographs; and
generating a report indicating a pattern of usage associated with one or more of the plurality of drug monographs based on the monitored activity of the plurality of users.

2. The method of claim 1, wherein the pattern of usage includes trend and occurrence information associated with one or more of the plurality of drug monographs.

3. The method of claim 1, wherein monitoring the activity of the plurality of users during the generation of the plurality of drug monographs comprises identifying a frequency of one or more studies being included, excluded, viewed, or not viewed in preparation of the plurality of drug monographs.

4. The method of claim 3, wherein the one or more studies include one or more client or competitor studies.

5. The method of claim 1, wherein monitoring the activity of the plurality of users during the generation of the plurality of drug monographs comprises identifying a geographic region associated with each user generating a drug monograph.

6. The method of claim 1, wherein generating the report indicating the pattern of usage associated with one or more of the plurality of drug monographs comprises indicating a pattern of usage associated with the healthcare information.

7. The method of claim 1, wherein the pattern of usage includes identifying a frequency of generation of drug monographs for a particular drug.

8. A server for generating healthcare information based on a plurality of drug monographs, the server comprising:
a processor; and
a memory comprising a plurality of instructions stored thereon that, in response to execution by the processor, causes the server to:
retrieve healthcare information from one or more databases;
generate a plurality of drug monographs using the healthcare information retrieved from the one or more databases and based on user input from a plurality of users;
monitor activity of the plurality of users during the generation of the plurality of drug monographs; and
generate a report indicating a pattern of usage associated with one or more of the plurality of drug monographs based on the monitored activity of the plurality of users.

9. The server of claim 8, wherein the pattern of usage includes trend and occurrence information associated with one or more of the plurality of drug monographs.

10. The server of claim 8, wherein to monitor the activity of the plurality of users during the generation of the plurality of drug monographs comprises to identify a frequency of one or more studies being included, excluded, viewed, or not viewed in preparation of the plurality of drug monographs.

11. The server of claim 10, wherein the one or more studies include one or more client or competitor studies.

12. The server of claim 8, wherein to monitor the activity of the plurality of users during the generation of the plurality of drug monographs comprises to identify a geographic region associated with each user generating a drug monograph.

13. The server of claim 8, wherein to generate the report indicating the pattern of usage associated with one or more of the plurality of drug monographs comprises to indicate a pattern of usage associated with the healthcare information.

14. The server of claim 8, wherein the pattern of usage includes identification of a frequency of generation of drug monographs for a particular drug.

15. One or more non-transitory machine-readable media comprising a plurality of instructions stored thereon that, in response to execution by a server, causes the server to:
retrieve healthcare information from one or more databases;
generate a plurality of drug monographs using the healthcare information retrieved from the one or more databases and based on user input from a plurality of users;
monitor activity of the plurality of users during the generation of the plurality of drug monographs; and
generate a report indicating a pattern of usage associated with one or more of the plurality of drug monographs based on the monitored activity of the plurality of users.

16. The one or more non-transitory machine-readable media of claim 15, wherein the pattern of usage includes trend and occurrence information associated with one or more of the plurality of drug monographs.

17. The one or more non-transitory machine-readable media of claim 15, wherein to monitor the activity of the plurality of users during the generation of the plurality of drug monographs comprises to identify a frequency of one or more studies being included, excluded, viewed, or not viewed in preparation of the plurality of drug monographs.

18. The one or more non-transitory machine-readable media of claim 17, wherein the one or more studies include one or more client or competitor studies.

19. The one or more non-transitory machine-readable media of claim 15, wherein to monitor the activity of the plurality of users during the generation of the plurality of drug monographs comprises to identify a geographic region associated with each user generating a drug monograph.

20. The one or more non-transitory machine-readable media of claim 15, wherein to generate the report indicating the pattern of usage associated with one or more of the plurality of drug monographs comprises to indicate a pattern of usage associated with the healthcare information.
